(19) United States
(12) Patent Application Publication
(10) Pub. No.: US 2008/0267928 A1
(43) Pub. Date: Oct. 30, 2008

(54) Compositions and Methods for Making Insulin-Producing Cells

(76) Inventor: Lijun Yang, Gainesville, FL (US)

Correspondence Address: EDWARDS ANGELL PALMER & DODGE LLP, P.O. BOX 55874, BOSTON, MA, 02205, US

(21) Appl. No.: 10/598,289

(22) PCT Filed: Feb. 23, 2005
(86) PCT No.: PCT/US05/05696

§ 371 Date: Jun. 26, 2007

Related U.S. Application Data
(60) Provisional application No. 60/547,006, filed on Feb. 23, 2004.

(51) Int. Cl. A61K 35/407; C12N 15/09; C12N 5/10

(52) U.S. Cl. 424/93.21; 435/455; 435/325

(57) ABSTRACT

An insulin-producing cell made by delivering a combination of at least two transcription factors, such as Pdx-1 and Pax4, to a liver cell. Cells according to the invention can be used to reduce an insulin insufficiency in a diabetic subject.

### FIELD OF THE INVENTION

The invention relates generally to the fields of developmental biology, stem cells, endocrinology, and medicine. More particularly, the invention relates to compositions and methods for making insulin-producing cells from liver cells.

### BACKGROUND

Recently published studies have demonstrated that hepatic stem cells could be induced in vitro to transdifferentiate into insulin-producing pancreatic endocrine-like cells. Other studies have shown that ectopic and transient expression of the transcription factor (TF), Pdx-1, in mouse liver induces transdifferentiation of hepatocytes into pancreatic cells including both exocrine and endocrine cells, and reduces hyperglycemia in chemically induced diabetic mice. While a promising strategy for treating type 1 diabetes, the conversion from liver to pancreas mediated by Pdx-1 unfortunately results in severe hepatitis due to production of by-products such as the exocrine enzymes, amylase and trypsin. Preliminary studies have also shown that under non-diabetic conditions, ectopic expression of Pdx-1 in mouse liver only converts hepatocytes into exocrine pancreas tissue, with no detectable increase in insulin gene or protein expression.

### SUMMARY

Methods of selectively making an insulin-producing cell from a liver cell have been developed. These methods involve delivering a combination of at least two TFs (e.g., Pdx-1 and Pax4) to a liver cell. By selecting the appropriate combination of TFs, a liver cell can be caused to selectively differentiate into insulin-producing cells, rather than into other types of pancreatic cells, such as exocrine pancreatic cells. The selectivity of this method is important in P-cell replacement therapies for treating diabetic subjects as it avoids undesired side effects such as hepatitis caused by undesired differentiation of a liver cell into a pancreatic exocrine cell.

The method includes the steps of: (a) providing a liver cell; and (b) increasing the level of TFs (e.g., Pdx-1 and Pax4) in the liver cell under conditions that promote the selective differentiation of the liver cell into an insulin-producing cell.

In another aspect, the invention includes a method of reducing an insulin insufficiency in a subject. This method includes introducing into the subject an insulin-producing cell made according to the foregoing method, or by introducing into the subject a composition that causes an increase in the level of TFs (e.g., Pdx-1 and Pax4) in one or more target (e.g., liver) cells in the subject.

Although Pdx1-VP16 expression induces hepatic cell transdifferentiation into pancreatic precursor cells, these incompletely reprogrammed cells fail to become into glucose-sensitive insulin-producing cells (IPCs) in the absence of late-stage transcription factors (TFs) (Cao et al Diabetes. 2004.53(12):3168-3178). In a preferred embodiment, hepatic cell lines are produced expressing Pax4 in the absence (WB-2 cells) or presence (WB-1A cells) of Pdx1-VP16 via lentiviral vector-mediated gene transfer. Preferably, activation of Pax4 results in the expression of the late-stage TFs including Pax6, Isl-1, and MafA, and generates the gene expression profile for WB-1A cells similar to the functional rat insulinoma INS-1 cells.

The term “subject,” as used herein, means a human or non-human animal, including but not limited to a mammal such as a dog, cat, horse, cow, pig, sheep, goat, chicken, primate, rat, and mouse.

The phrase “liver cell” as used herein means any cell found in the liver of an animal at any stage of development from embryo to adult.

Unless otherwise defined, all technical terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety for the proposition cited for. In the case of conflict, the present specification, including any definitions will control. In addition, the particular embodiments discussed below are illustrative only and not intended to be limiting.

### BRIEF DESCRIPTION OF THE DRAWINGS

The invention is pointed out with particularity in the appended claims. The above and further advantages of this invention may be better understood by referring to the following description taken in conjunction with the accompanying drawings, in which:

FIG. 1A-I is a series of scanned photomicrographs showing the results of an immunohistochemical analysis of liver sections.

FIG. 2 is a graph showing the effects of CMV-Pdx-VP16 pDNA on blood glucose in Stz-induced diabetic mice.

FIG. 3 A-D is a series of scanned photomicrographs showing liver stem cell regeneration in a liver sections.

FIG. 4 is a graph showing the effects of Pdx-VP16 on blood glucose in Stz-induced diabetic DDC mice.

FIG. 5 is a highly schematic illustration of a lentiviral transduction system. “A”, shows Components of the system, while “B” shows SIN vector 3′ U3 promoter.

FIG. 6 A-F is a series of phase and fluorescence scanned micrographs illustrating the transduction of hepatic cells with Lenti-eGFP.

FIG. 7 A-D is a series of scanned images relating to the generation and characterization of the WB-1 cell line. A. Fluorescence micrographs of WB cells were transfected with plasmids containing Pdx-1-VP16 and RIP-eGFP genes. The single-cell-derived positive clone (WB-1) was selected (A) and expanded (B and C). The resulting cell cluster expressed the insulin gene as indicated by cytoplasmic GFP expression (D). B. Expression profiles of genes related to beta cell development. C. Detection of Pdx-1 protein expression by Western blot. D. Immunocytochemistry with anti-Pdx-1 antibody.

FIG. 8 is a graph showing blood glucose levels in animal subjects. 1×06 cells/mouse of WB (control, n=3), WB-1 (n=6), or INS-1 (n=4) cells were implanted under the left subrenal capsule after the blood glucose levels reached 400 mg/dl (arrow, TX-transplanted) in repeatedly low-dose STZ-treated mice. The left kidney of three mice from WB-1 group was removed at 40 days post implantation (arrow, Ex-explanted).

FIG. 9 is a comparison of gene expression profiles between (pre Tx) and (post Tx) transplanted WB-1 cells. Upper long arrow indicates the proposed sequence of the transcription factor cascade in developing endocrine pancreas. Explanted tissue (Post Tx) containing implanted WB-1 cells expressed three late stage genes (Pax4, Pax6, and Isl-1, arrowheads) that were silent in Pre-Tx WB-1 cells. Arrows indicate genes with relatively increased expression compared to Pre-Tx. Ngn3 expression (arrow) was undetectable in Post-Tx cells.

FIGS. 10 A-F is a series of scanned photomicrographs of histological sections showing insulin production in explanted tissue. The explanted tissue (A. lower power) shows a rich microvascular network and glandular and islet-like structures (B. higher power). Insulin immuno-staining in an islet (C) of the pancreas serves as positive control and insulin staining of the explanted tissue (E) shows the majority of cells expressing cytoplasmic insulin in contrast to negative control (D) stained with guinea pig serum.

FIG. 11 is two graphs showing insulin content (A) and insulin release (B) in WB and WB-1 cells. Cells were stimulated with 20 mM glucose for 2 h and various culture media as indicated in B were collected for insulin releaseassay. Cells then were washed three times and lysed with lysis buffer to obtain cell lysates for insulin content measurement. Insulin in the cell lysates and the media was detected by ELISA. WB-1 cells after long-term culture in a high glucose medium exhibited the ability of production (A) and release of insulin (B) in response to a glucose.

FIG. 12 are scanned images showing gene expression profiles in WB-1 and WB-1A cells. Introduction of Pax4 gene into the Pdx-1-VP16 expressing WB-1 cells by LV transduction generates the WB-1A cells that coexpressed both Pdx-1-VP16 and Pax4 genes. The gene expression profiles between WB-1 and WB-LA were compared and showed that: 1) activation of Pax4 (arrow) resulted in the activation of Pax6, Isl-1, and MafA genes (arrows), which were silent in WB-1 cells; and 2) overexpression of Pax4 in WB-1A cells down-regulated Nkx2.2 expression (arrow). The profile of gene expression in WB-1A cells is similar to that in the posttransplanted WB-1 cells as well as to that in INS-1 cells.

FIG. 13 is a schematic representation of a method of generating specified cell lines.

FIG. 13B shows the scanned image results from the method used for the transduction of WB cells with LV-GFP. To evaluate the transduction efficiency, WB cells were transduced with LV-GFP at a MOI of 20 for 48 hrs and the transduction efficiency is nearly 100% (GFP-positive cells).

FIGS. 14A and 14B are scanned Western blots showing the expression of Pdx1 and Pdx1-VP16 proteins. Cell lysates extracted from WB-1, WB-1A, WB-2, WB, and INS-1 cells were separated by SDS-PAGE. Pdx1 & Pdx1-VP16 proteins were detected by Western blotting with anti-Pdx1 (FIG. 14A) antibody (1:5000, C. V. Wright) and anti-VP16 (FIG. 14B) antibody (1:200, BD). Arrowheads indicated two forms of Pdx1 proteins and Pdx1-VP16 fusion proteins (phosphorylated upper and dephosphorylated lower bands, respectively).

FIGS. 15 A and 15B show comparison of scanned gene expression profiles among the WB-derived cell lines (FIG. 15A and FIG. 15B). Total RNA was extracted from the cells and RT-PCR was performed. All primers (Table 1) were designed across intron(s). INS-1 (clone 823/13) cells as β-cell positive control. Short-arrows indicate exogenous (mPdx1) and endogenous (rpdx1) expression in WB-1 & WB-1A cells. Long-arrows indicate exogenous Pax4 expression. Arrowheads indicate newly activated genes. Star indicates the position of down-regulated Nkx2.2. Certain lanes contain two bands and the lower bands represent the primer dimer.

FIG. 16A shows scanned images showing the detection of insulin and glucagon by immunocytochemistry. Slides made from newly generated WB-1 cells, WB-1A, WB-2, WB cells, and INS-1 cells were stained with anti-insulin (1:500) and glucagon (1:100) antibodies. Insulin staining was shown in red, glucagon in green, and nuclei in blue (Dapi). Rat pancreatic islet and INS-1 cells served as positive controls. FIG. 16B scanned images showing shows the detection of insulin-secretory granules by EM. Globular ultrastructure (upper left) and immunogold-labeled insulin secretory granules (arrows, lower left) were detected in cultured WB-LA cells. Rat islet β-cell served as positive control (right). N represents nucleus, and G indicates globular secretory vesicles.

FIG. 17A is a graph showing blood glucose levels from transplanted cells. WB, WB-1, WB-1A, or WB-2 cells (1×10⁶/mouse) were implanted under the left subrenal capsules of Stz-treated diabetic NOD-scid mice (arrow, Tx=transplantation). The blood glucose levels were monitored under nonfasting condition. The left kidney from the transplanted mice was removed around day-40 post-Tx (arrow, Ex=explanation). All pancreata from these mice were examined for islet cell regeneration by H&E & insulin immunostaining. FIG. 17B shows scanned images showing the histology and insulin production in the explanted WB-1A cells. H&E (upper) and insulin staining (1:500) (lower) were performed on paraffin sections of the explanted WB-1A cells (day-42 post-Tx). Arrows indicate the renal subcapsular space filled with transplanted WB-1A cells. Pancreas served as controls. FIG. 17C shows scanned images showing the insulin production among the explanted cells. Diabetic NOD-scid mice received WB-derived (WB, WB-2, WB-1, or WB-1A) cells. These explanted tissues were stained with anti-insulin antibodies (1:500). Pancreatic islet served as positive controls (Right panel). Right upper corner represents H&E-stained mouse pancreas.

FIG. 18 is a schematic representation of transcription factor cascade in liver-to-endocrine pancreas transdifferentiation.

### DETAILED DESCRIPTION

The invention provides methods of making cells that secrete insulin by increasing the level of a combination of at least two (Transcriptional Factors) TFs in liver cells. By selecting the appropriate combination of TFs, liver cells can be caused to selectively differentiate into insulin-producing cells rather than into other types of pancreatic cells (e.g., exocrine pancreatic cells). The cells made by these methods can be used to reduce insulin insufficiency (e.g., caused by diabetes) in a subject by introducing the cells into the subject or by introducing into the subject a composition that causes an increase in the level of TFs in one or more target (e.g., liver) cells in the subject.

### Biological Methods

Methods involving conventional molecular biological techniques are described herein. Such techniques are generally known in the art and are described in detail in methodology treatises. Molecular biological techniques are described in references such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Conventional methods of gene transfer and gene therapy can also be adapted for use in the present invention. See, e.g., Gene Therapy Methods: ed. M. I. Phillips, Vol. 436, Methods in Enzymology, Academic Press, 2002; Gene Therapy: Principles and Applications, ed. T. Blackenstein, Springer Verlag, 1999; Gene Therapy Protocols (Methods in Molecular Medicine), ed. P. D. Robbins, Humana Press, 1997; and Retro-vectors for Human Gene Therapy, ed. C. P. Hodgson, Springer Verlag, 1996.

### Making Insulin-Producing Cells From Liver Cells

Methods of the invention utilize liver cells as source cells from which insulin-producing cells can be made. Any type of liver cell capable of being differentiated into an insulin-producing cell may be used, e.g., a mature hepatocyte or an hepatic stem cell. Depending on the particular application of the invention, the liver cell can be from a particular species, e.g., a human being, a rat, a mouse, or other another mammal. In the examples described below, liver epitheleal cells derived from normal liver were used.

WB-F344 rat liver epithelial cells (WB cells) were used in the experiments described below. These are normal liver cell line isolated from an adult male Fisher 344 rat and represent the cultured counterpart of liver stem-like cells. They express hepatocyte markers such as albumin, tyrosine aminotransferase, and alpha-1-antitrypsin and are capable of differentiating into both mature hepatocytes and biliary epithelial cells. In making the invention, several stably transfected Pdx-1-VP16 positive rat WB cell lines containing a reporter gene of RIP-eGFP were made by plasmid transfection with lipofectin.

Mature hepatocytes might also be used in some aspects of the invention. These cells, however, are difficult to maintain and expand in cell culture. To enhance their ability to replicate in culture, primary human hepatocytes can be transduced with the catalytic subunit of telomerase.

### TFs Involved In Pancreatic Endocrine Development

The differentiation and maturation of the endocrine islet cells during development is a complex process controlled by a unique network of gene regulation. Of the different TFs, Pdx-1 is thought to have the greatest likelihood for encoding the difference between liver and pancreas. A modified, activated form of Pdx-1 termed Pdx-1-VP16 (VP16 derived from herpesvirus) is especially active in stimulating differentiation along a pancreatic pathway. As mentioned above, expression of Pdx-1 in a liver cell promotes differentiation of the cell towards both a pancreatic exocrine and endocrine phenotype. To selectively promote differentiation towards only an endocrine phenotype, other TFs can be used in combination with Pdx-1.

Other TFs that are expressed selectively in the endocrine pancreas in the developing pancreas and that could play a role in endocrine cell fate decisions have been identified. These factors all contain homeodomains and can be divided into early factors (Pax4, Nkx2.2, and Nkx6.1) that are coexpressed with neurogenein3 (Ngn3) in endocrine progenitor cells, and later factors (Pax6, isl1, Bm4, HB9, and Pdx-1) that are found in more mature cells. In the experiments described below, Pax4 in combination with Pdx-1 caused selective differentiation of liver cells into an insulin-producing cells but not into pancreatic exocrine cells. Thus the invention contemplates using multiple TFs (2, 3, 4, 5 or more) to selectively convert liver cells into endocrine pancreas, e.g., insulin-producing β-like cells.

In the studies described below, introduction of Pdx-1-VP16 fusion gene alone into WB liver cell line (WB-1 cells) activated multiple genes related to pancreas development but did not promote the production and release insulin. However, when the cells were transplanted into diabetic mice, reduction of hyperglycemia due to insulin production was observed, indicating that a diabetic microenvironment is important for the complete conversion into insulin-producing cells. When the Pax4 gene was introduced into WB-1 cells that expressed the Pdx-1-VP16 fusion gene, the cells demonstrated a gene expression profile similar to the functional beta cell line INS-1 cells, and subsequently become glucose-responsive, functional liver-derived insulin-producing cells.

### Other Factors For Promoting β Cell Development

In addition to a suitable combination of TFs, other soluble factors can contribute to the selective conversion of hepatocytes to endocrine pancreas. These include glucagon like peptide (GLP-1), Exendin-4, betacellulin, activin A, and islet neogenesis-associated protein (INGAP), nutrients such as glucose, and other factors such as nicotinamide.

Glucose in particular has been shown to play a critical role in the differentiation of cells toward a β-cell phenotype. To promote the differentiation of a cell into an insulin-secreting cell the cell is preferably placed in a high glucose environment, e.g., greater than about 9 mM (e.g., 8.9, 9, 10, 11, 12, 13, 14, 15, 16, 17, 18, 19, 20, 21, 22, 23, 24, 25, 26, 27, 28, 29, and 30 mM) for a sufficient period of time, e.g., at least about several days (e.g., 55, 60, 65, 70, 75, 80, 85, 90, 100 or more days). A cell can be transferred from a low glucose to a high glucose to promote differentiation. Maturation of the cell might sometimes be achieved by then returning the cells to a low glucose environment. See, e.g, Yang et al., Proc. Matl. Acad. Sci. USA 99: 8078-8083, 2002.

### Increasing The Level of TFs in a Target Cell

Various methods of the invention include the step of increasing the level of TFs in a liver cell to promote its differentiation to an insulin-producing cell. This step can be achieved by introducing into the cell a composition that causes an increase in the level of TFs (e.g., Pdx-1 and Pax4) in the liver cell. This method can be employed where the liver cell is in an in vitro culture or when it is located in situ in an animal subject. The composition that causes an increase in the level of TFs can take the form of a cocktail of the TF proteins themselves which can be contacted or injected into the cells. It may also take the form on an agent that acts to increase expression of genes encoding or regulating expression of the TFs, e.g., small molecule drugs. For convenience, however, the composition that causes an increase in the level of TFs preferably takes the form of one or more nucleic acids encoding the TFs, e.g., in a gene delivery vector.

Many gene delivery systems have been well studied for their pros and cons for gene- and cell-based therapeutics for type I diabetes, including plasmid DNA (pDNA), adenovirus (Ad), recombinant adeno-associated virus (rAAV), MLV-based retrovirus (MLV), and lentivirus (LV). Among these, pDNA is generally easier to engineer, grow and purify, while the advantages of rAAV are that integration is stable and site-specific, and many cell types can be transduced. For MLV, a major limitation is low transduction efficiency when targeting nondividing cells. LV has been shown to transduce both proliferating and nondividing cells and cell lines in vitro with near 100% of transduction efficiency and permanently integrate into host cell genome without generating immunogenicity, in contrast to Ad and rAAV. It is now considered to be the most promising vector system in future gene therapy. Recent studies have demonstrated successful in vitro transduction of mouse, rat and human primary hepatocytes in culture or in suspension with LV, and when transplanted into the liver of the recipient animals, these LV-transduced hepatocytes extensively repopulate the liver and remain differentiated and functional hepatocytes for up to a year. In making the invention, several lentiviral vectors containing Pdx-1, Pdx-1-VP16, RIP-eGFP, Pax4 genes were prepared. Rat and human hepatic cells were successfully transduced with Lenti-eGFP with 100% of efficiency.

It has been known for years that naked DNA can be delivered to cells in vivo and result in gene expression. Intravascular delivery of pDNA is very effective to transfer genes into of hepatocytes. A major advance in the intravascular delivery of pDNA was the recent development of the tail vein injection procedure to rapid delivery of a relatively large volume (10% of the body weight of a mouse or rat) within 5 to 7 s time frame into the mouse and 15-20 s into rat. The tail vein drains into the inferior vena cava. Delivery of a large bolus into tail vein in a very short time presumably results in the blood volume in the vena cava backing up due to the volume being too large for the heart to handle rapidly. Blood backs up predominantly into the liver through the hepatic vein into the system of terminal hepatic veins (central veins) that are in direct contact with hepatocytes, resulting in gene transfer into hepatocytes. Tail vein or hydrodynamic-based gene delivery has been shown to result in very high levels of gene transfer, typically 10 to 15% of the hepatocytes in mouse liver but levels up to 40% have been reported. By this approach, transgene expression is also found in heart, spleen, and kidney, at levels about 100-fold lower than liver. Due to its simplicity and reproducibility, the tail vein injection has been adopted remarkably quickly in the gene therapy field for basic research and gene therapy evaluation. Since the liver is a major target organ for the methods of the invention and is also a major organ transfected by this means, tail vein injection is a preferred method for delivering genes into hepatocytes in rodents.

### Method of Reducing a Insulin Insufficiency

In yet another aspect, the invention provides a method of reducing insulin insufficiency in a subject. This method may be performed by introducing into the subject a composition including (a) insulin-producing cells made by increasing the level of TFs in live cells as described herein or (b) an agent that causes an increase in the level of TFs in one or more target (e.g., liver) cells in the subject.

Suitable subjects for use in the invention can be any animal. For example, the subject can be an animal such as mammal like a human being, dog, cat, horse, cow, pig, sheep, goat, chicken, primate, rat, or mouse. Preferred are subjects suspected of having or at risk for developing a disorder of insulin insufficiency, e.g., a person suspected of having or at risk for developing type I diabetes, based on clinical findings or other diagnostic test results.

The cells/compositions of the invention can be administered to animals or humans by any conventional technique. Such administration might be parenteral (e.g., intravenous, subcutaneous, intramuscular, or intraperitoneal introduction). Preferably, the cells/compositions may also be administered directly to the target site (e.g., to the liver, pancreas, renal subcapsular space or skin) by, for example, surgical delivery, such as implantation to an internal or external target site, or by catheter to a site accessible by a blood vessel. Implantation of cells may include inserting implantable cellular delivery systems that permit release of secreted insulin, but prevent destruction of the insulin-producing cells by the immune cells of the host.

A preferred method of introduction of the cells of the invention may be by techniques currently in use for transplantation of islet cells recovered from the pancreata of human cadavers. See, e.g., Shapiro A J M et al., N Engl J Med 343:230-238, 2000. In this method, islets (or cells) are delivered under local anaesthesia, by x-ray fluorographic guidance of a long thin needle, into the portal vein of the liver. Once in the portal circulation, the islet cells enter the portal spaces and take up residence, becoming surrounded by new blood vessels. The rich blood supply in the vicinity of the transplanted cells promotes effective secretion of hormones directly into the blood stream.

An effective number of insulin-producing cells sufficient for reducing or eliminating insulin insufficiency can be determined by established procedures for evaluation of outcomes of pancreatic islet cell transplantation. In general, determination of an effective amount of the composition is made using standard methods known in the art, such as measurement of blood glucose levels in the subject before and after administration of the cells/compositions.

The source of the liver cells used to produce the insulin-producing cells that are introduced into the subject can either be autologous or heterologous. The option of producing autologous cells from the subject presents an attractive alternative to a regimen of lifelong immunosuppressive therapy to control the risk of rejection of the introduced cells. Autologous insulin-producing cells can be prepared, as described above, by obtaining a liver biopsy from the subject by routine procedures, and propagating the liver cells contained within the liver biopsy, to produce insulin-producing cells.

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

All publications and patent documents cited in this application are incorporated by reference in pertinent part for all purposes to the same extent as if each individual publication or patent document were so individually denoted. By their citation of various references in this document, Applicants do not admit any particular reference is “prior art” to their invention.

### EXAMPLES

### Example 1

### Ectopic Expression of Pdx-1-Vp16 Fusion Gene in Normal Mice after Hydrodynamics-Based Tail Vein Delivery of pDNA Converts Hepatocytes into Pancreatic Exocrine Cells

To determine if ectopic expression of Pdx-1 in the liver can convert hepatocytes into pancreatic tissue, 16 non-obese diabetic (NOD) male mice, which do not spontaneously develop diabetes, were anesthetized and injected through the tail vein with 40 μg naked pDNA of CMV-Pdx-1-Vp16 (n=8), or empty vector (n=8) in 2 ml saline within 7 seconds. One injected mouse from each group was sacrificed on day 1, 2, 3, 5, 7, 17, 47, and 60. The tissues of liver, kidneys, heart, spleen, and pancreas with duodenum were harvested either in formalin and embedded in paraffin for morphologic evaluation and immunohistochemicstry, or snap-frozen with liquid nitrogen for analysis of gene expression and tissue insulin and amylase protein production. Snap frozen liver tissue from each mouse was used to extract total RNA with Trizol reagent and 1 μg of total RNA from each sample was used for RT-PCR or a real-time RT-PCR analysis for gene expression of Pdx-1, insulin, and amylase. The production of Pdx-1, insulin, and amylase proteins was detected by immunohistochemistry using specific antibodies.

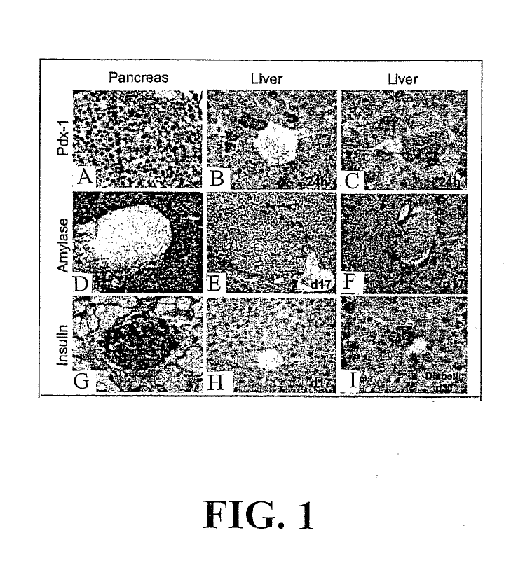

FIGS. 1A, D, and G show pancreatic expression of Pdx-1 (β cells), amylase (exocrine cells) and insulin (β cells) as positive controls, respectively. FIGS. 1B and C show ectopic expression of Pdx-1 protein in the liver (5-10% of liver cells) detected by anti-Pdx-1 antibody 24 hr after injection of CMV-Pdx-1-VP16 plasmid. There was no significant Pdx-1 expression at later time points. Liver sections from mice injected with empty vector revealed no Pdx-1 protein expression. In the liver of mice receiving injection of CMV-Pdx-1-VP16 plasmid, amylase protein was first detected at day 17 (FIGS. 1E and F). The amylase positive cells were orderly distributed along the central vein region and secreted amylase into the bile canaliculi (FIG. 1F, arrow). However, insulin and glucagon hormone production was not detectable in the liver of the mice injected with Pdx-1-VP16 and collected at all time points (FIG. 1H, a representative picture of insulin staining at day 17). In addition, focal patchy necrosis next to the central veins was present at early time points (FIGS. 1B and C) and these injuries were repaired quickly and completely disappeared at day 7. This patchy necrosis is most likely due to the high pressure of tail vein injection since it was also present in the liver of mice receiving an empty vector injection. Mice sacrificed after day 7 showed no obvious pathologic changes such as hepatitis, necrosis or cirrhosis in the liver. Real time RT-PCR studies demonstrated that Pdx-1 gene expression peaked at the end of day 1, and was reduced the expression after day 2. However, the gene expression of the Pdx-1 was persistent at low levels in the liver of CMV-Pdx-1-VP16 injection mice throughout the observed period (60 days). No Pdx-1 expression was observed in the vector control group. Amylase gene expression in the liver tissue was detected beginning on day 5 and was continuously present through the observation period. However, the gene expression of insulin and glucagon could not be detected even at 35 cycles of RT-PCR. A total RNA from mouse pancreas was used as a positive control for RT-PCR studies.

These studies indicate that hydrodynamics-based gene delivery of Pdx-1-VP16 pDNA effectively introduces Pdx-1-VP16 gene into the hepatocytes and results in 5-10% of hepatocytes expressing Pdx-1 protein. However, the Pdx-1 expression in the liver cells is transient and quickly becomes undetectable by immunohistochemistry. Under the nondiabetic conditions, the Pdx-1-positive liver cells subsequently were converted into amylase-producing pancreatic cells but there was no clear evidence of expression of either insulin gene or protein, suggesting that the ectopic overexpression of Pdx-1 in liver cells in non-diabetic mice does not automatically convert the liver cells into pancreatic endocrine cells.

### Example 2

### Hydrodynarnics-Based Transfection of Pdx-1-Vp16 Fusion Gene in Streptozotocin (stz)-Induced Diabetic Mice

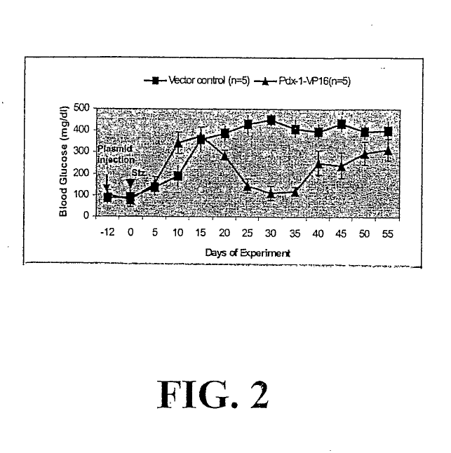

To determine whether the diabetic microenvironment such as hyperglycemia may facilitate Pdx-1 positive hepatocyte transdifferentiation into pancreatic endocrine hormone producing cells, NOD male mice were induced to become diabetic by an intraperitoneal injection (ip) of streptozotocin, an antibiotics that can selectively destroy pancreatic β cells, (200 μg/g) 12 days after receiving Pdx-1-VP16 (n=5) or empty vector (n=5) through tail vein injection. The blood glucose levels were monitored every five days (FIG. 2). The mice receiving empty vector became hyperglycemic with glucose levels above 350 mg/dl (normal 70-100 mg/dl) within 10 days (solid square line). However, the blood glucose levels in mice receiving CMV-Pdx-1-VP16 slowly increased for a few days and then decreased to near normal range (100-150 mg/dl) (triangle line). The glucose levels of the mice receiving Pdx-1-VP16 became low for a while (d25-d35), and then went back up (˜300 mg/kg) until the end of the experiments. On day 55 after stz treatment, the mice were sacrificed and tissue from the liver and pancreas from each mouse was collected for the analysis of gene expression and pancreatic hormone production as described above.

RT-PCR analysis showed the presence of Pdx-1, amylase, and insulin gene expression in mice injected with CMV-Pdx-1-VP16 pDNA but not with empty vector control. Immunohistocychemistry revealed rare insulin-positive cells, mainly around the central vein (FIGS. 1-I, d30). Amylase staining cells were rare.

These studies show that hyperglycemia in diabetic mice facilitates the transdifferentiation of Pdx-1 positive liver cells into insulin-producing cells but the effects appear to be short lived and transient; lasting a couple of weeks. The pattern of blood glucose changes, presumably due to changes in insulin secretion in the diabetic mice could be explained by 1) low yield of transfection of the hepatocytes by pDNA, 2) in vivo premature silence of the CMV promoter, and 3) transfection of mature hepatocytes by Pdx-1-VP16 generating differentiated pancreatic cells with a short life span.

### Example 3

### Hydrodynamics-Based Transfection of Pdx-1-Vp16 Fusion Gene in Diabetic DDC Mice

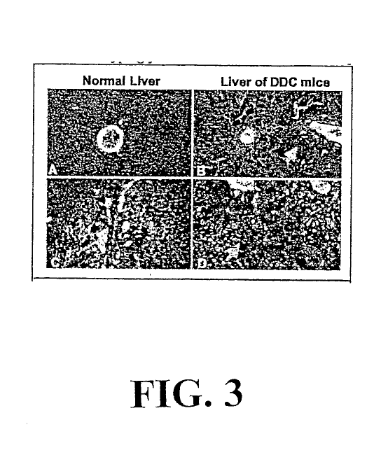

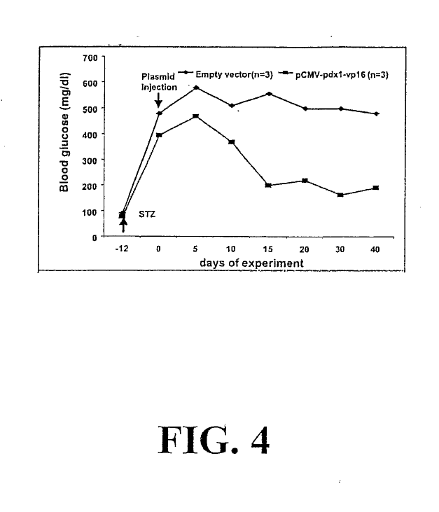

To determine whether transfection of hepatic stem cells could induce long-lasting effects in normalization of blood glucose levels in diabetic mice, Pdx-1-VP16 was injected into the tail vein of mice with a model of DDC-induced liver cell injury. NOD male mice were fed with a liver toxin DDC diet for 30 days and liver sections from both normal and DDC mice were examined for liver stem cell regeneration as shown in FIG. 3. Sections of normal liver (A and C) and liver from DDC-treated mice (B and D) revealed a marked increase in small dark liver stem cells (oval cells) with a high nucleus to cytoplasm ratio and these cells were distributed along the bile ducts as indicated by arrows (B and D). Oval stem cells specific marker (A6) immunostaining highlighted these stem cells (C and D). The mice were subsequently injected with streptozotocin (25 μg/g) every other day for three times to induce hyperglycemia. The dose of streptozotocin was reduced due to severely compromised liver functions. Within 12 days after the first streptozotocin injection, the glucose levels of these mice were elevated above 350 mg/dl. The mice with hyperglycemia then received a tail vein injection of 30 μg plasmids of CMV-Pdx-1-VP16 (n=3) or empty vector (n=3). The blood glucose levels were monitored every five days. Analysis of blood glucose levels in these mice (FIG. 4) demonstrated a significant, persistent reduction of blood glucose levels in mice receiving Pdx-1-VP16 in contrast to the control vector mice, suggesting that transfection of the hepatic stem cells with Pdx-1-VP16 may exhibit a long lasting reduction of the blood glucose levels.

### Example 4

### Construction and Generation of Plasmid DNA

CMV-Pdx-1 (Neor), RIP-eGFP (zeor), CMV-Bata2/NeuroD (Neor), and CMV-Pax4 (Neor) plasmids were generated for in vivo tail vein injection and in vitro transfection studies. The plasmids of TTR-Pdx-1-VP16:Elas-GFP, and CMV-Pdx-1-VP16 (Neor) were kindly provided by Marko Horb (Bath University, United Kingdom). The human telomerase construct was provided by Dr. Chen Liu (Pathology, University of Florida). Other plasmids containing full length cDNA of Pdx-1, NeuroD/beta2, Ngn3 and Pax4, NKx2.2, and Nk6.1 were obtained from Christopher V. Wright, Hsiang-Po Huang, and Michael S. German, respectively.

### Example 5

### Generation of Lentiviral Vectors

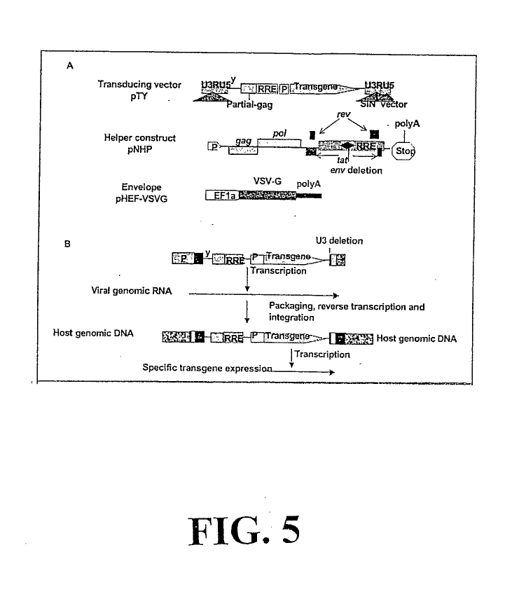

A high-titer, safety- and efficiency-improved lentiviral vector system that has broad host cell tropism has been developed. The pTY-based vector has extensive deletions including deletion in the viral long terminal repeats (LTPs) and thus does not carry an active viral promoter after infection (self-inactivating vector, SIN, FIG. 5A). The lentiviral SIN vectors can accommodate up to 8 kb of foreign gene insertion, and when co-transfected into 293T cells with two helper plasmids, pNHP (encoding viral capsid and enzymes) and pHEF-VSVG (encoding viral envelop), will produce replication-defective vectors with titer higher than 10⁹ infectious units/ml after concentration. Different from many other vector systems, lentiviral vectors permanently integrate into the host cell genome after infection (FIG. 5B). Lentiviral gene expression occurs in less than 24 h after infection and the transgene expression is permanent due to viral integration. Lentiviral vectors encoding Pdx-1, Pdx-1-VP16, RIP-eGFP, Pax4, and human telomerase were generated using the SIN pTY-based vectors. The cDNA of the above target genes under the control of CMV promoter were cloned into the polylinker region of pTYF (see FIG. 5A). The lentiviral helper construct pNHP, and pHEFVSVG, and the transducing vector were co-transfected into 293T cells to produce lentiviral particles as previously described. Zaiss et al., J. Virol., 76:7209, 2002. The concentration of vectors were normalized by real-time RT-PCR according to Sastry et al. Gene Ther., 9:1155, 2002. The vectors were concentrated by ultracentrifugation or filtration. The titers of the Lenti-vectors reach 109 transducing units/ml after concentration. The concentrated vectors were aliquoted, and stored at −80° C. until use.

### Example 6

### Transduction of Hepatic Cells with Lenti-eGFP

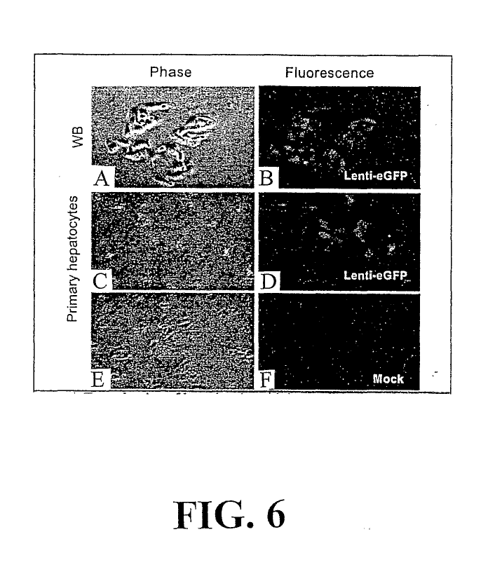

Primary hepatocytes (PH) were isolated from NOD-scid mice by collagenase digestion and cultured for 12 h in DMEM containing 25 mM glucose and supplemented with 10% FBS, 100 units/ml penicillin, 100 μg/ml streptomycin, 5 μM hydrocortisone, and 5 μg/ml insulin. To determine the transduction efficiency of lenti-vectors for hepatic cells, WB cells and PH were transduced with lenti-eGFP by exposing cells to lenti-eGFP for 48 hours at a multiplicity of infection (MOI) of 10 and 50. All transductions were performed in modified growth medium containing 8 ug/ml polybrene. 293T cells were used as positive control for the transduction procedure. For negative control (mock infection), cells were incubated in the same medium in the presence of empty LV. Nearly 100% efficiency of transduction was observed in WB cells with lenti-GFP vector using a MOI of 10 for 48 hr. More than 95% of PH expressed GFP using a MOI of 50 for 48 h. FIG. 6 shows representative images of expression of eGFP in WB cells and PH. There was no fluorescence signal detected in control cells, PH (Mock infection).

### Example 7

### Immortalization of Primary Human Hepatocyte (PHH) with Lenti-Telomerase and Transduction of Hepatic Cells with Lenti-eGFP

PHH were isolated from healthy liver tissue obtained from surgical liver wedge biopsy specimens collected after informed consent from patients undergoing diagnostic or therapeutic biopsy or partial hepatectomy according to the Institutional Review Board guideline. The cells were isolated by collagenase digestion and cultured in DMEM containing 25 mM glucose and supplemented with 10% FBS, 100 units/ml penicillin, 100 μg/ml streptomycin, 5 μM hydrocortisone, and 5 μg/ml insulin. The PHH cells were immortalized by a lentiviral vector containing the catalytic subunit of the human telomerase gene (PHH-hTERT). The immortalized PHH-HTERT cells continuously grow and can undergo more than 50 passages without overt evidence of senescence. To determine the transduction efficiency of lenti-vectors for hepatic cells, WB cells and PHH-hTERT were transduced with lenti-eGFP by exposing cells to lenti-eGFP for 48 hours at a multiplicity of infection (MOI) of 50. All transduction were performed in modified growth medium containing 8 ug/ml polybrene. 293T cells were used as positive control for transduction procedure. For negative control (mock infection), cells were incubated in the same medium in the presence of empty lentiviral vector. Nearly 100% efficiency of transduction was observed in all hepatic cell lines with lenti-GFP vector using a multiplicity of infection (MOI) of 50 for 48 hr. FIG. 6 shows representative images of expression of eGFP in WB cells and PHH-hTERT. There was no fluorescence signal detected in control cells, PHH-hTERT (Mock infection).

### Example 8

### Generation and Characterization of Stably Transfected WB Cell Line

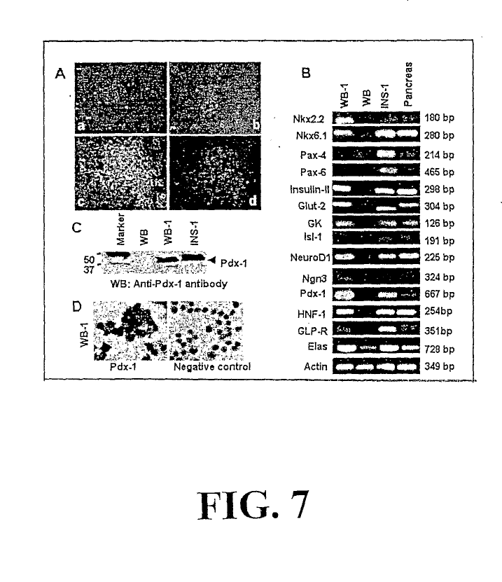

To establish an in-vitro system to study the molecular mechanism of the liver to pancreas transdifferentiation, stably transfected WB cells line containing genes of both CMV-Pdx-1-VP16 and RIP-eGFP were generated. Rat WB cells were first transfected with CMV-Pdx-1-VP16 plasmid and selected with antibiotics G418 (400 μg/ml) for four weeks. A single cell-derived cell clone (FIG. 7A-a) was isolated, further expanded (A-b), and then transfected with RIP-eGFP in a zeocin resistant plasmid by lipofectin and double selected with both antibiotics G418 (400 μg/ml) and Zeocin (400 μg/ml) for three weeks. Single cell derived stably transfected WB cell clones (A-c) expressing both genes were selected using a cloning cylinder (Fisher Scientific, Pittsburgh, Pa.) and named the WB-1 cell line. These cells have a built-in reporter gene RIP-eGFP, which generates green fluorescence when the insulin gene is expressed (A-d). The WB-1 cells were characterized by examining gene expression by RT-PCR (FIG. 7B), confirming the overexpression of Pdx-1 protein by Western blot (FIG. 7C) and the presence of Pdx-1 protein in the nuclei of WB-1 cells by immunocytochemistry (FIG. 7D) using an anti-Pdx-1 antibody (Christopher V. Wright). FIG. 7B shows the comparison of gene expression profiling among WB-1 cells, parent WB cells, rat insulinoma INS-1 cells (C. Newgard, Duke Univ.), and rat pancreas. The results show that WB-1 cells exhibit a similar profile of gene expression to that seen in the rat P-cell line INS-1 cells and rat pancreas, except for the absence of gene expression of Pax4, Pax6 and Isl-1 in WB-1 cells, suggesting that WB-1 cells are precursor cells of endocrine pancreas. The WB-1 cells were insensitive to glucose stimulation with no significant detectable insulin release by ELISA (ALPCO Diagnostics) when the cells were stimulated with 20 mM glucose for 2 hr. Absent expression of Pax4, Pax6, and Isl-1 genes, which are critical in the late stage of P-cell differentiation and in maintaining mature β cell function, may explain the insensitivity of glucose stimulated insulin release in WB-1 cells on culture without P-cell stimulating factors.

### Example 9

### Transplantation of WB-1 Cells into Nod/Scid Diabetic Mice

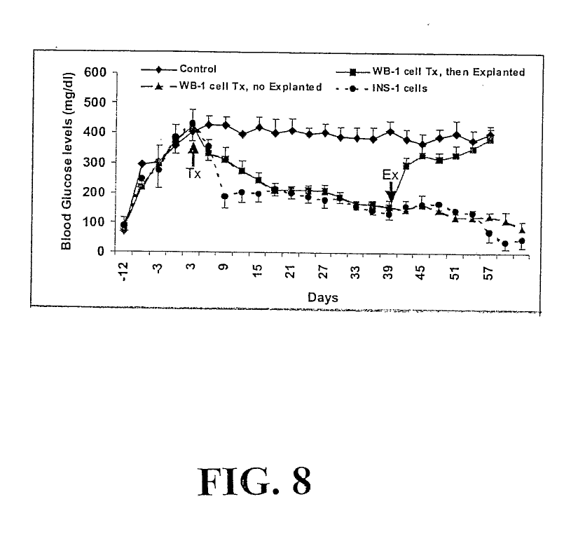

To test whether an in vivo diabetic environment can promote pancreatic precursor cells to further differentiate and mature, WB-1 cells were transplanted into diabetic NOD/scid mice. Parent WB cells were used as a negative control and rat insulinoma cells (INS-1) were used as a positive control. The normalization of blood glucose levels and restoration of the weight loss in diabetic mice are key endpoints for evaluation of the efficacy of cellular therapy for type 1 diabetes mellitus (T1DM). As demonstrated in FIG. 8, WB-1 cells can reduce hyperglycemia significantly after 2-3 weeks under the in vivo diabetic condition and eventually lead to complete reversal of the diabetes associated hyperglycemia and weight loss, indicating that the implanted cells are fully functional in vivo. The pattern of reduction of the blood glucose levels with WB-1 cells is similar to INS-1 cells except those ES-1 cells show a rapid effect on glucose reduction. It generally takes 5-7 days for implants to build up capillary networks to survive in the new environment. The difference between the initial part of the curves reflects the time needed for WB-1 cell differentiation and maturation. In contrast, no time is needed for fully functional INS-1 cells. Removal of implanted WB-1 cells (n=3) by left nephrectomy resulted in a rebound persistent hyperglycemia indicating that the implanted WB-1 cells are indeed responsible for the reduction of blood glucose levels. WB cells had no effect on the blood glucose levels. Towards the end of the observation period, INS-1 cells caused much lower blood glucose levels (˜40 mg/dl) in nonfasting mice; whereas, WB-1 cells maintained a perfect normoglycemia (˜70 to 90 mg/dl). This may be explained by in vivo uncontrolled INS-1 cell proliferation. These results indicate that the pancreatic precursor WB-1 cells require in vivo diabetic environment to further differentiate and mature to become fully functional beta-like cells.

### Example 10

### Comparison of Gene Expression Between Pre- and Post-Transplanted WB-1 Cells

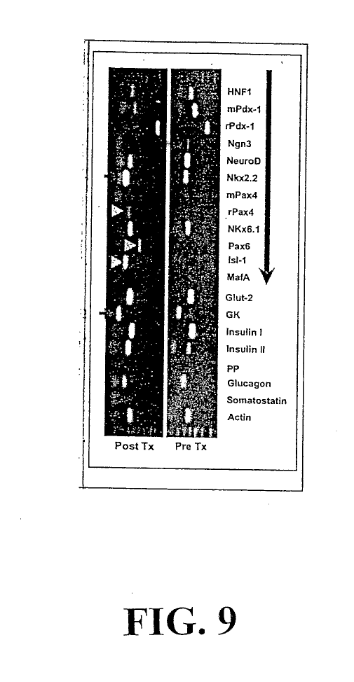

To explore the molecular mechanism of the shift of WB-1 cells from being nonfunctional (glucose insensitive) in vitro to being functional in vivo, total RNA was extracted from the explanted tissue containing WB-1 cells and compared the expression profiles of certain key genes between pre- and post-transplanted WB-1 cells by RT-PCR. As shown in FIG. 9, several noticeable changes were demonstrated in the activation of the β cell developing genes: 1) the explanted cells, after 40 days in vivo under the renal capsule, expressed Pax4, Pax6, and Isl-1 (light arrow heads), all three late stage genes in the β cell development, which were not expressed in the pretransplanted WB-1 cells; 2) gene expression of Ngn3, a key transcription factor that is transiently expressed in the pancreatic endocrine precursors, but not in mature pancreatic endocrine cells, became undetectable as compared to pre-transplanted WB-1 cells (long arrow); and 3) there was a noticeable increase in NK2.2, GK, and Insulin II gene expression levels in post transplanted cells (other arrows), as compared to the levels of pretransplanted cells. These results indicate that in vivo diabetic conditions (e.g. hyperglycemia) play a key role for the pancreatic precursor (WB-1) cells to further differentiate and mature to become functional pancreatic endocrine cells. The molecular landmarks of functional insulin-producing β-like cells may be defined by the inactivation of Ngn3 gene and activation of late stage genes as mentioned above.

From these experiments, activation of Nkx6.1 gene appears to be in a higher hierarchy than the activation of Pax4 gene in contrast to the proposed position for Pax4 in the developing pancreas. Therefore, it is clear that Pdx-1 alone as well as its direct targets cannot directly activate late stage genes (Pax4, Pax6, and Isl-1). Activation of these late stage genes in the liver and endocrine pancreas conversion can be achieved by placing the precursor cells into a diabetic environment in mice.

### Example 11

### Histologic Structure and Insulin Production of WB-1 Cells in the Explanted Tissue

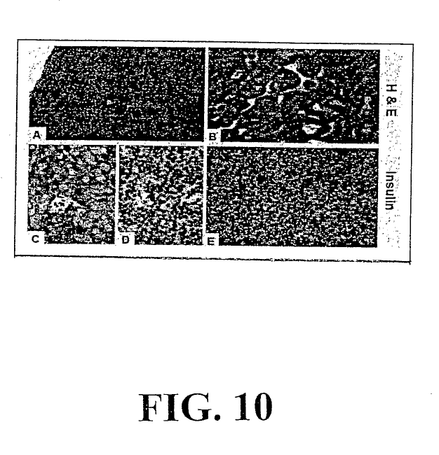

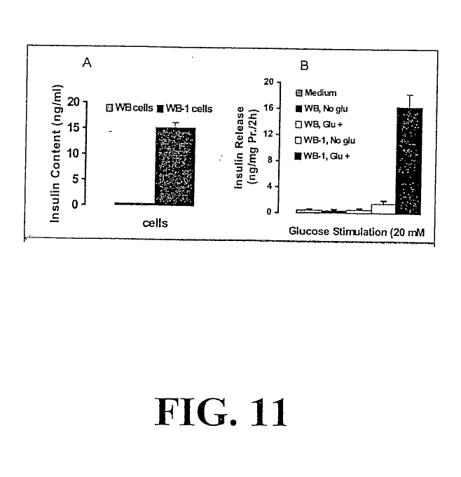

To determine the histological appearance and pancreatic hormone production after in vivo maturation, the explanted tissue was harvested in formalin fixative and embedded in paraffin for morphologic evaluation and immunohistochemical staining with insulin, glucagon, and amylase antibodies. As shown in FIG. 10, H&E stained section (A and B), the implanted WB cells form glandular or islet-like clusters with a rich network of microvasculature to ensure the survival of the transplants. The lower panel shows immunostaining with insulin antibody (C, positive control of islet, D negative control with guinea pig serum, and E insulin in explanted tissue). The large majority of cells in the explanted tissue expressed insulin (E). Only scattered cells expressed glucagon and there were no detectable levels of amylase protein production. These results suggest that the in vivo differentiation and maturation effectively convert pancreatic precursor cells into mature pancreatic endocrine cells. However, residual hepatic features and function remain to be determined. The possible explanations for the in vivo maturation of the implanted cells may include that 1) A diabetic microenvironment such as hyperglycemia promote immature cell differentiation and maturation; and 2) Subrenal capsular implantation creates cell-cell contact in a three-dimensional environments to allow cell-extracellular matrix interaction promoting cell maturation and producing insulin.

### Example 12

### Effects of High Glucose on Promoting In Vitro WB-1 Cell Differentiation

The newly derived WB-1 cell line showed insulin gene expression by RT-PCR but no detectable insulin release by ELISA. To determine whether long-term high glucose culture can induce the WB-1 cells to undergo further differentiation, the culture of WB-1 cells was continued in medium containing a high glucose concentration (11 mM) for another two months. As demonstrated in FIG. 11, WB-1 cells after long-term culture in a high glucose medium exhibited the ability of production (A, insulin content) and release of insulin (B. insulin release) in response to a glucose challenge (20 mM for 2 hr). In contrast, WB cells showed no insulin production and insulin release. Although there was glucose-responsive insulin release from WB-1 cells, a small amount of insulin leaking into the culture medium without glucose stimulation (3 mM glucose medium) was noted—a phenomenon that was also observed in a β cell line insulinoma INS-1 cells.

### Example 13

### Generation of Plasmid DNA and Lentiviral Vectors

CMV-Pdx-1 (Neor), RIP-eGFP (zeor), and CMV-Pax4 (Neor) plasmids for in vitro cell transfection were generated. The plasmids of TTR-Pdx-1-VP16:Elas-GFP, and CMV-Pdx-1-VP16 (Neor) were kindly provided by Marko Horb (Bath University, United Kingdom). The plasmids containing full length cDNA of Pdx-1, were obtained from Christopher V. Wright. LVs encoding Pdx-1, eGFP, RIP-eGFP, Pdx-1-VP16, and Pax4 were generated using the SIN pTY-based vectors. In brief, the cDNA of the above target genes under the control of CMV promoter were cloned into the polylinker region of pTYF. The lentiviral helper construct pNHP, and pHEFVSVG, and the transducing vector were co-transfected into 293T cells to produce lentiviral particles as previously described. Zaiss et al., supra. The concentration of vectors were normalized by real-time RT-PCR according to Sastry et al., supra. The vectors were concentrated by ultracentrifugation or filtration. The titers of the LVs reach 109 transducing units/ml after concentration. The concentrated vectors were aliquoted, and stored at −80° C.

### Example 14

### Comparison of Gene Expression Between WB-1 and WB-1A Cells

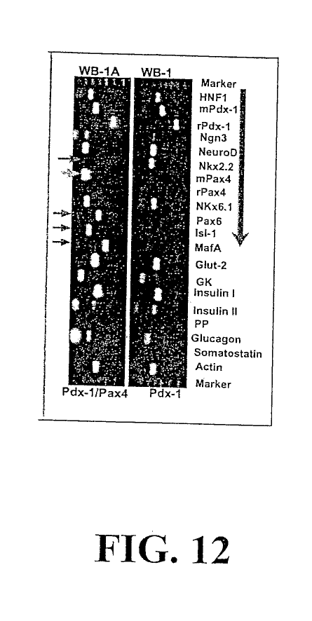

In WB-1 cells, expression of Pdx-1-VP16 activated many upstream genes of developing endocrine pancreas, but not the late stage genes Pax4, Pax6, Isl-1, and MafA. Pax4 is positioned at a higher hierarchy in the map of TFs in β cell development. To determine the effects of activation of Pax4 gene in WB-1 cells (Pdx-1-VP16+) on gene expression profiles, WB-1 cells were transduced with LV containing CMV-Pax4 gene, called WB-1A. The gene expression of WB-1 and WB-1A cells was detected by RT-PCR. As demonstrated in FIG. 12: 1) introduction of exogenous mouse Pax4 (light arrow) into WB-1 cells resulted in the activation of Pax6, Isl-1, and MafA genes (darker arrows), which were silent in WB-1 cells; 2) overexpression of Pax4 in WB-LA cells inhibited Nkx2.2 expression (arrow), possibly by a negative feedback (Nkx2.2 is positioned immediately upstream of Pax4 in β cell development); and 3) the profile of gene expression of WB-LA cells is almost identical to that in INS-1 cells and is similar to that in the post-transplanted WB-1 cells as shown in FIG. 9, suggesting a more mature profile of gene expression in WB-LA cells. Indeed, WB-1A cells demonstrate in vitro glucose-responsive insulin release measured by ELISA. These findings indicate that transduction of hepatic cells with a combination of TFs of Pdx-1-VP16 and Pax4 produces a gene expression profile most close to functional pancreatic β cell line and this combination may be the most effective means to transdifferentiate hepatocytes into functional IPCs.

### Example 15

### Pdx1- and Pax4-Mediated Liver-to-Endocrine Pancreas Transdifferentiation

Abbreviations: ChromA, chromogranin-A; GK, glucokinase; GLP-1R, glucagon-like peptide 1 receptor; Glut-2, glucose transporter 2; HK, hexokinase; HNF1, hepatocyte nuclear factor 1; IAPP, islet amyloid polypeptide protein; Isl-1, transcription factor islet-1; Kir6.2, inward rectifier K (+) channel member 6.2; MafA, basic leucine-zipper transcription factor; NeuroD/Beta2, the basic helix-loop-helix transcription factor, a key regulator of both insulin gene transcription and neurogenic differentiation; Ngn3, neurogenin 3; NKx2.2, NK2 transcription factor related, locus 2; NKx6.1, NK6 transcription factor related, locus 1; NOD-scid, nonobese diabetic severe combined immunodeficient; RIP-eGFP, rat insulin-1 promoter-enhanced green fluorescent protein; Stz, streptozotocin; Pax4, the paired homeodomain gene 4; Pax6, the paired homeobox domain gene 6; Pdx1, pancreatic duodenal homeobox 1; Pdx1-VP16, a fusion gene of Pdx1 with the activation domain of VP16 from Herpes simplex virus; PC1/3, prohormone convertase 1/3; PC2, prohormone convertase 2; SNAP25, 25 kDa synaptosomal associated protein; SUR1, sulphonylurea receptor 1.

### Materials and Methods

Plasmid constructs. A Pdx1-VP16 expression vector was constructed by fusing the coding sequence for the eighty residue VP16 activation domain to the C-terminus of mouse Pdx1. The RIP-eGFP plasmid was constructed as described elsewhere (Cao, L Z, Tang, D Q, Horb, M E, Li, S W, Yang, L J: High Glucose is Necessary for Complete Maturation of Pdx-1-VP16-Expressing Hepatic Cells into Functional Insulin-Producing Cells. Diabetes 53(12):3168-3178, 2004). Lentiviral vector (LV) containing Pax4 or eGFP genes (Stratagene La Jolla, Calif.) was constructed by inserting the entire mouse Pax4 coding sequence (Smith, S B, Ee, H C, Conners, J R, German, M S: Paired-homeodomain transcription factor PAX4 acts as a transcriptional repressor in early pancreatic development. Mol. Cell Biol. 19:8272-8280, 1999) or eGFP into the pTYF vector cassette under control of the elongation factor-1α(eEF-1α) promoter. Lentivirus was produced and the titer determined.

Transduced cell lines. Rat liver epithelial WB cells, representing the cultured counterpart of liver stem-like cells, were derived from normal liver cells from an adult male Fisher 344 rat. WB-1 cells (expressing both Pdx1-VP16 and RIP-eGFP genes) were generated from WB cells by stable transfection with the Pdx1-VP16 fusion gene and a RIP-eGFP reporter gene. WB-2 cells (expressing Pax4 alone) were generated by transducing WB cells with LV-Pax4 vector. WB-1A cells (expressing both Pdx1-VP16 and Pax4 genes) were generated by transducing WB-1 cells with LV-Pax4. Cell transduction by LV was conducted as previously described (Chang, L J, Zaiss, A K: Lentiviral vectors. Preparation and use. Methods Mol.Med. 69:303-318, 2002; Chang, L J, Urlacher, V, Iwakuma, T, Cui, Y, Zucali, J: Efficacy and safety analyses of a recombinant human immunodeficiency virus type 1 derived vector system. Gene Ther. 6:715-728, 1999). Transduction efficiency was determined by transducing WB cells with L V-eGFP at a multiplicity of infection of 20.

Culture conditions. WB-derived cell lines were maintained in RPMI 1640 medium supplemented with 10% FCS and 11.1 mM D-glucose. The rat INS-1 cell line (clone 832/13) (gift from Christopher B. Newgard) has been maintained continuously for one and one-half years in RPMI 1640 medium and served as a positive control for rat β-cells.

RT-PCR. Total RNA was prepared from cells using TRI-reagent, and gene expression was determined by RT-PCR. The forward and reverse PCR primers (see Table 1 for exact sequences employed) were designed to be located in different exon(s).

ELISA. Insulin content in cell lysates and insulin release into the culture medium from the WB, WB-1, WB-1A, and WB-2 cells were preformed in triplicate.

Western blotting. Pdx1 and Pdx1-VP16 fusion proteins were detected according to our previously published methods. In brief, cell lysates (50 μg/lane) were separated by SDS-PAGE using 12% Tris-HCl gels (Bio-Rad). Proteins were transferred and blotted with rabbit anti-Pdx1 serum (1:5000), followed by HRP-conjugated secondary anti-rabbit polyclonal antibody (1:20,000). Proteins were visualized by ECL.

Cell transplantation. Male NOD-scid mice (8-10 weeks old) were made hyperglycemic by ip injections of streptozotocin (Stz) at 50 μg/g body weight daily for five days, as previously described (Tang, D Q, Cao, L Z, Burkhardt, B R, Xia, C Q, Litherland, S A, Atkinson, M A, Yang, L J: In vivo and in vitro characterization of insulin-producing cells obtained from murine bone marrow. Diabetes 53:1721-1732, 2004). When blood glucose levels reached >350 mg/dL, mice were transplanted with WB-1, WB-1A WB-2, or WB-GFP cells (1×10⁶ cells/mouse) into the left renal capsular space. The blood glucose levels were monitored regularly at 16:00 in non-fasting condition. Transplanted cells were removed by left nephrectomy around day-40 post-transplantation to assess metabolic activity and morphologic characteristics. The control hyperglycemic mice were terminated around day-40 post-transplantation.

Histology and immunohistochemistry. The explanted tissues containing implanted WB, WB-2, WB-1, and WB-1A cells from transplanted mice were fixed and embedded in paraffin, and sections were then stained with hematoxylin and eosin (H&E). Sections were incubated with anti-insulin antibodies (1:500, Dako).

Immunofluorescence. Slides were prepared from cultured WB, WB-1, WB-1A, and WB-2 cell lines, fixed with methanol for 10 min, and incubated with antibodies directed against insulin (1:500) or glucagon (1:100). DAPI was used to highlight cell nuclei. Mouse pancreas tissue and INS-1 cells served as positive controls for insulin and glucagon staining.

Electron microscopy (EM) and inmunogold antibody labeling. Cell ultrastructure and immunogold insulin localization in cultured WB-LA cells and rat pancreas were performed as previously described (Cao, L Z, Tang, D Q, Horb, M E, Li, S W, Yang, L J: High Glucose is Necessary for Complete Maturation of Pdx-1-VP16-Expressing Hepatic Cells into Functional Insulin-Producing Cells. Diabetes 53(12):3168-3178, 2004).

Statistical analysis. The statistical significance of our experimental findings was analyzed by using an independent sample t test, using a P value of less than 0.05 for the data to be considered significant.

Results

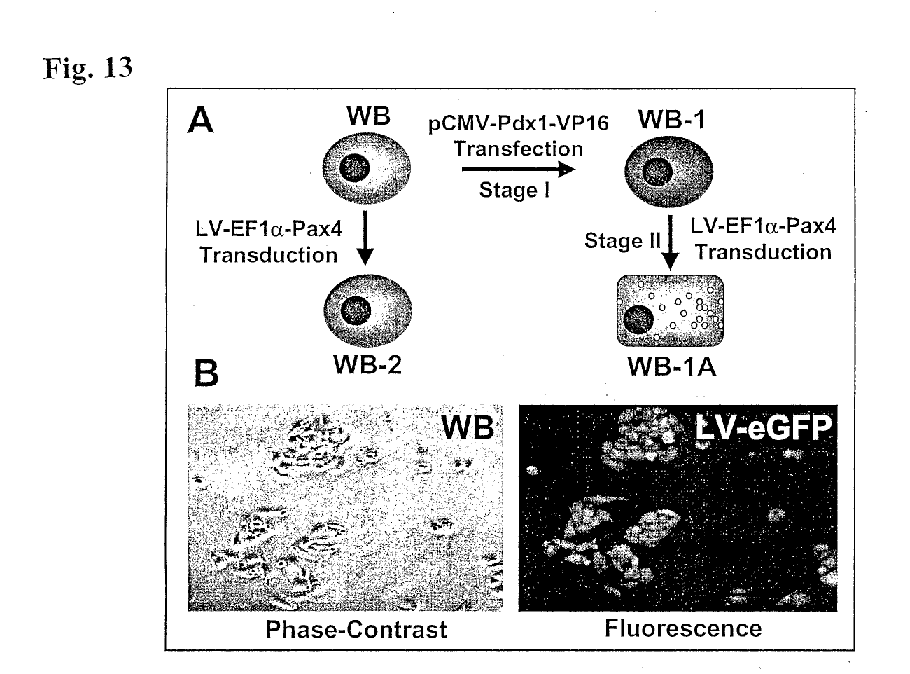

Generation of WB-derived cell lines. Pdx1-VP16 expression in hepatic stem-like WB cells results in their transdifferentiation into β-cell precursor-like cells (Cao, L Z, Tang, D Q, Horb, M E, Li, S W, Yang, L J: High Glucose is Necessary for Complete Maturation of Pdx-1-VP16-Expressing Hepatic Cells into Functional Insulin-Producing Cells. Diabetes 53(12):3168-3178, 2004). The latter cells can mature into functional IPCs upon exposure in vivo to a diabetic hyperglycemic microenvironment or upon culturing at high glucose concentration. However, the newly generated WB-1 cells did not express late-stage Pax4, Pax6, Isl-1, and MafA genes normally appearing during β-cell development; they also failed to release insulin in response to in vitro glucose stimulation. To study the role of Pax4 in the Pdx1-VP16-mediated P-cell transdifferentiation, we first developed several WB-derived cells lines that express Pdx1-VP16, Pdx1-VP16/Pax4, Pax4, or GFP genes. The WB-1 cell line (expressing Pdx1-VP16) was derived by co-transfection with CMV-Pdx1-VP16/neo^{r} and RIP-eGFP/zeor plasmids and selection of double-positive single cell clone. We then generated WB-1A and WB-2 cells by transducing WB-1 or WB cells with LV-Pax4, respectively (FIG. 13A). The activation of Pax4 in WB-1 cells allows us to study role of Pax4 in the Pdx1-VP16-mediated liver-to-β-cell transdifferentiation, and introduction of Pax4 into WB cells allows us to determine a role of Pax4 alone in the liver-to-p-cell transdifferentiation. WB cells transduced with LV-GFP served as controls for lentiviral vector and for evaluation of transduction efficiency. As demonstrated by counting GFP-positive WB cells at 48 hr after L V-GFP transduction (MOI of 20), the transduction efficiency was nearly 100% (FIG. 13B), and no further selection of positive single-cell clone was needed for the purpose of our intended studies.

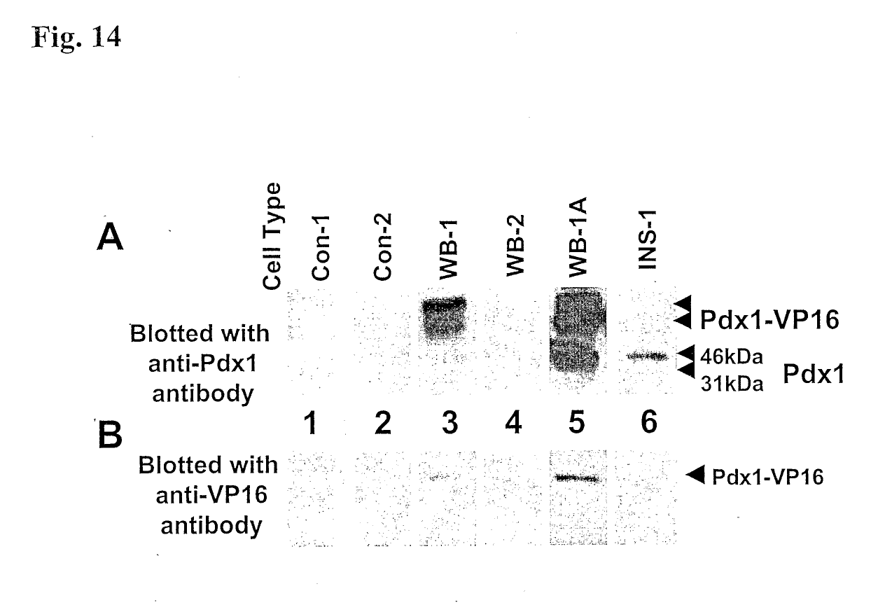

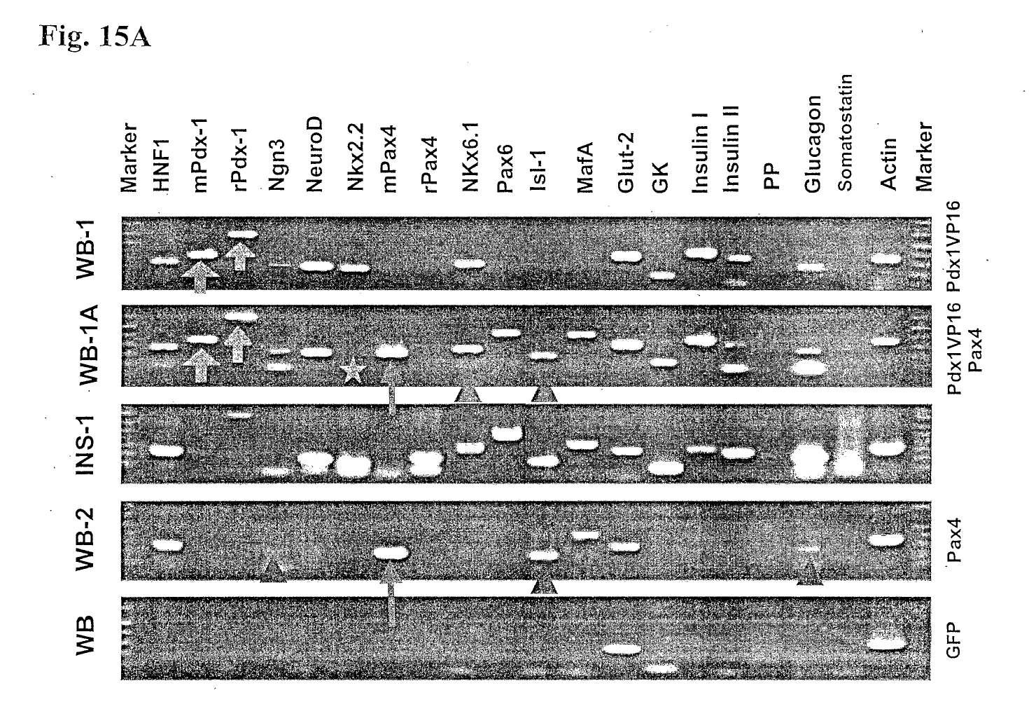

Pdx1 and Pdx1-VP16 protein expression. To confirm that the cell lines expressed Pdx1 and its fusion protein Pdx1-VP16, the proteins of interest were detected by Western blotting with anti-Pdx1 (FIG. 14A) and anti-VP16 (FIG. 14B) antibodies following separation of the whole cell lysates (50 μg/lane) on 12% gels. As expected, both WB-1 and WB-1A cells expressed the fusion protein of Pdx1-VP16 (Lanes 3 and 5, FIG. 14A top two bands & FIG. 14B) as well as weak bands of endogenous Pdx1 in WB-1 cells (Lane 3). Interestingly, activating Pax4 in WB-1 cells resulted in high-level expression of endogenous Pdx1 proteins (strong 46 kDa band and weak 31 kDa band). This is consistent with the pattern of a mature β-cell Pdx1 expression that was observed in functional rat insulinoma INS-1 cells (Lane 6, only loading 5 μg cell lysates). No Pdx1 or Pdx1-VP16 bands were detected in either WB-2 cells (Lane 4) or plasmid-transfected (Lane 1) or L V-GFP-transduced (Lane 2) control WB cells. These results indicated that activation of Pax4 in WB-1A cells up-regulated the expression of endogenous activated forms of Pdx1.

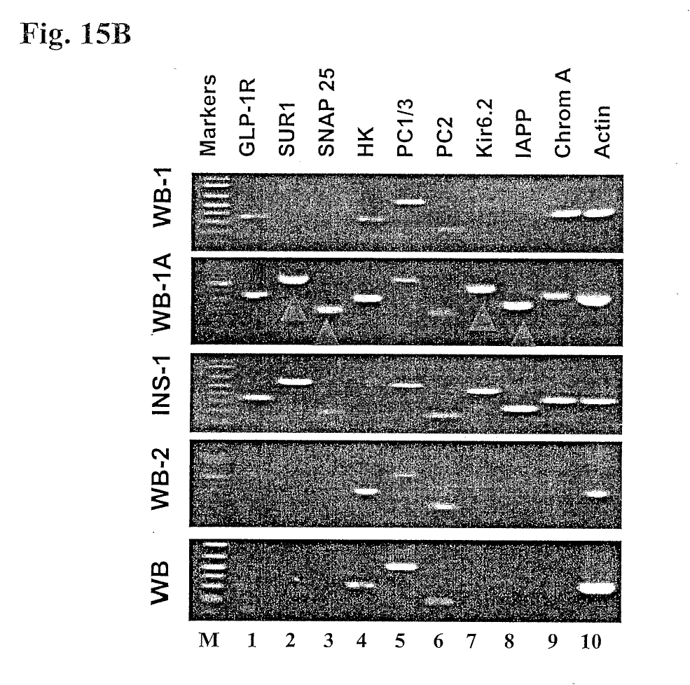

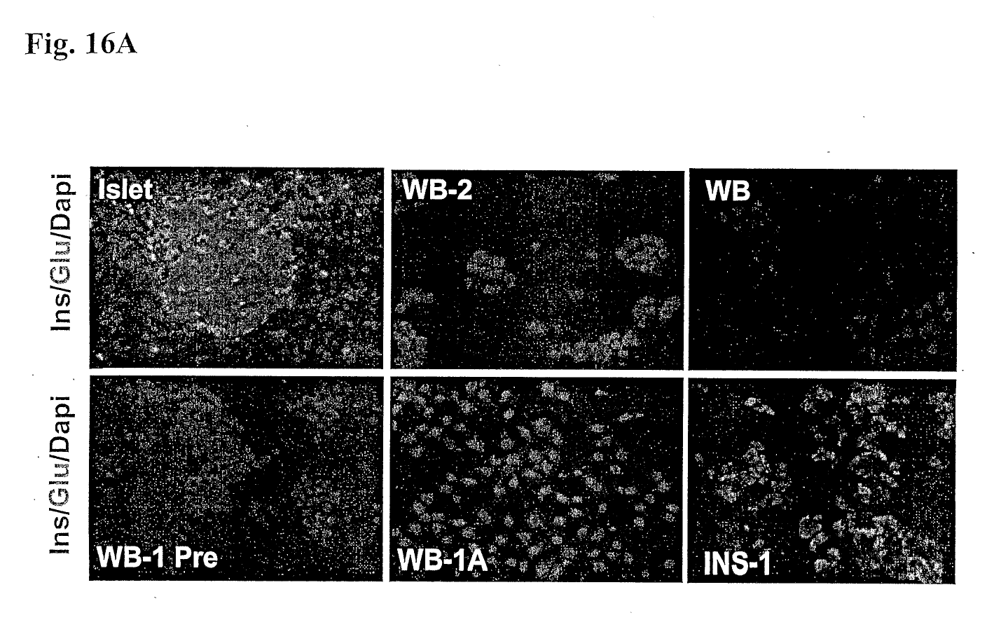

Comparison of gene expression profiles. Gene expression profile in newly generated WB-1 cells resembles pancreatic endocrine precursor cells. The cells expressed HNF1, endogenous Pdx1, Ngn13, NeuroD/Beta2, NKx2.2, and NKx6.1 as well as numerous genes related to pancreatic endocrine function (insulin I & II, glucagon, Glut-2, GK, GLP-1R, PC1/3, PC2, HK, and Chromo A). However, in WB-1 cells, Pdx1-VP16 was unable to activate the late-stage pancreatic transcription factors Pax-4, Pax-6, MafA, and Isl-1, as well as β-cell function-related genes SUR1, Kir6.2, SNAP25, and IAPP without further differentiation. Pax4 expression has been proposed to occur after NKx2.2 expression during the development of pancreatic β-cells. To determine the role of Pax4 in the liver-to-endocrine pancreas transdifferentiation, and to explore the underlying molecular mechanisms of this process, we compared the gene expression profiles among WB-1, WB-1A, WB-2, WB-GFP, and INS-1 cells by RT-PCR (FIGS. 15 A and 15B). As shown in FIGS. 15A and 15B, following Pax4 activation (long arrows) in WB-1A cells, the genes (Pax-6, Isl-1, and MafA) related to late-stage P-cell development (FIG. 15A) and genes (SUR1, SNAP25, Kir6.2, and IAPP) related to β-cell function and insulin secretion (FIG. 15B) were activated (arrowheads).

The gene expression profile for WB-1A cells resembles that of functional P-cell insulinoma INS-1 cells, and post-transplanted WB-1 cells, suggesting a mature P-cell gene expression profile in WB-1A cells. Moreover, Pax4 overexpression in WB-A cells significantly down-regulated Nkx2.2 expression (indicated by star), possibly by a negative feedback mechanism, since Nkx2.2 is positioned immediately upstream of Pax4 in P-cell development. In contrast, Pax4 alone in WB-2 cells only activated Ngn3, Isl-1, MafA, and glucagon, indicating that these genes may be direct targets of Pax4, and activation of Pax-6, SUR1, Kir6.Z SNAP25, or IAPP appears to require active participation of other pancreatic transcription factors.

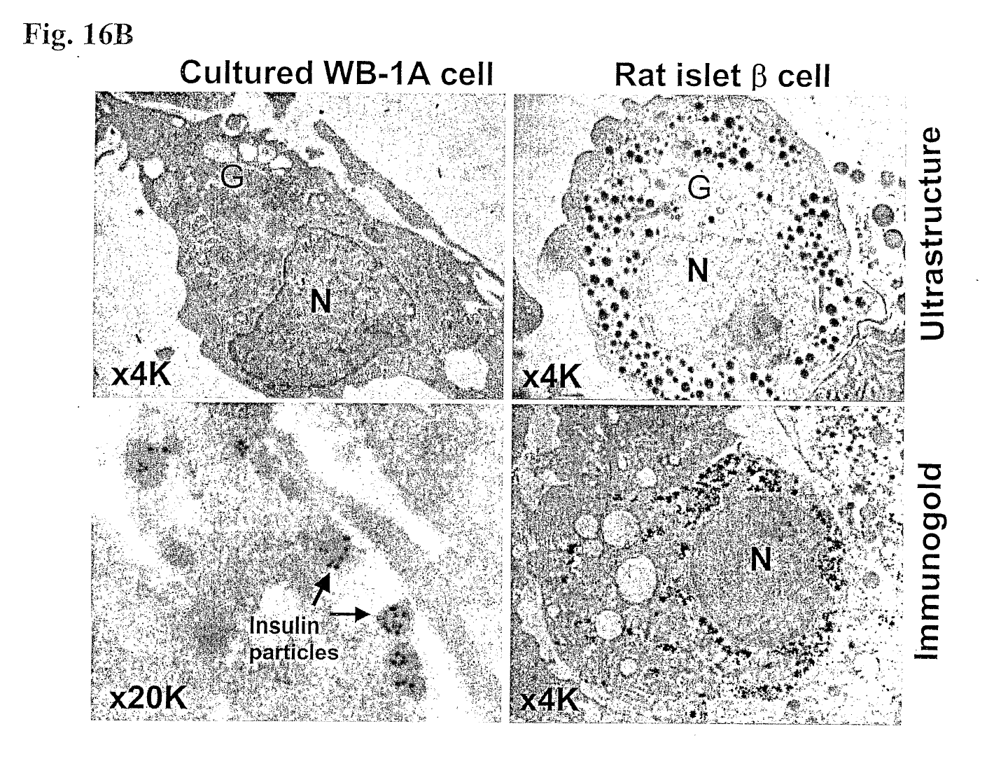

Insulin protein expression. Given the distinct patterns of Pdx1 protein expression as well as gene expression pattern between WB-1 and WB-1A cells, the levels of expression of insulin and glucagon were compared by immunocytochemistry (FIG. 16A). Cells were stained with anti-insulin and anti-glucagon antibodies and corresponding fluorescent-dye-labeled secondary antibodies. WB-1A cells showed intense cytoplasmic insulin staining (red), while WB-1 cells (without further differentiation) exhibited very weak insulin staining. No insulin was detected in WB-2 and WB-GFP cells. In contrast, glucagon-staining cells were not seen in any of the examined cells. Rat pancreatic islet (FIG. 16A, upper left) served as positive controls for insulin-positive β-cells (red) and glucagon-positive a-cells (green).

To examine whether insulin secretory vesicles were present in the liver-derived IPCs, EM was performed using immunogold-labeled antibody to detect insulin on the WB-1 and WB-1A cells. FIG. 16B show the ultrastructure of cultured monolayer WB-1A cells (upper left) with scattered globular structures and electron-dense insulin granules were detected by immunogold labeling (FIG. 16B, lower left, arrows). Pancreatic β-cell from rat pancreas served as positive controls. No insulin granules were detected in the newly generated WB-1 cells (data not shown); however, after in vitro differentiation insulin granules were detected in matured WB-1 cells.

In vitro functional characterization. To determine whether W-1A cells are functional, in vitro insulin release upon glucose challenge was examined. The cells were stimulated with 20 mM D-glucose medium for 2 hours after overnight incubation of the cells with 0.5% FCS in a low glucose (3.0 mM) medium. The culture media and cell lysates were collected and subjected to ELISA for insulin. Table 2 summarizes the insulin content and release in WB, WB-1, WB-1A, and WB-2 cells upon glucose stimulation. These results indicate that the WB-1A cells not only contained abundant insulin (8× greater than in WB-1 cells) but also responded to a glucose challenge, as evidenced by releasing insulin. The 2× increase in insulin release into the medium after glucose stimulation is statistically significant (P<0.001). However, the newly generated WB-1 cells were incapable of releasing insulin upon glucose stimulation and contained very low insulin levels. No insulin synthesis or release was detected in WB and WB-2 cells. These results indicate that Pax4 activation is necessary for promoting the Pdx1-VP16-expressing cells to synthesize, process, and release insulin in a glucose-responsive manner. Furthermore, activation of Pax4 alone is not sufficient to activate insulin expression and to convert liver cells into IPCs.

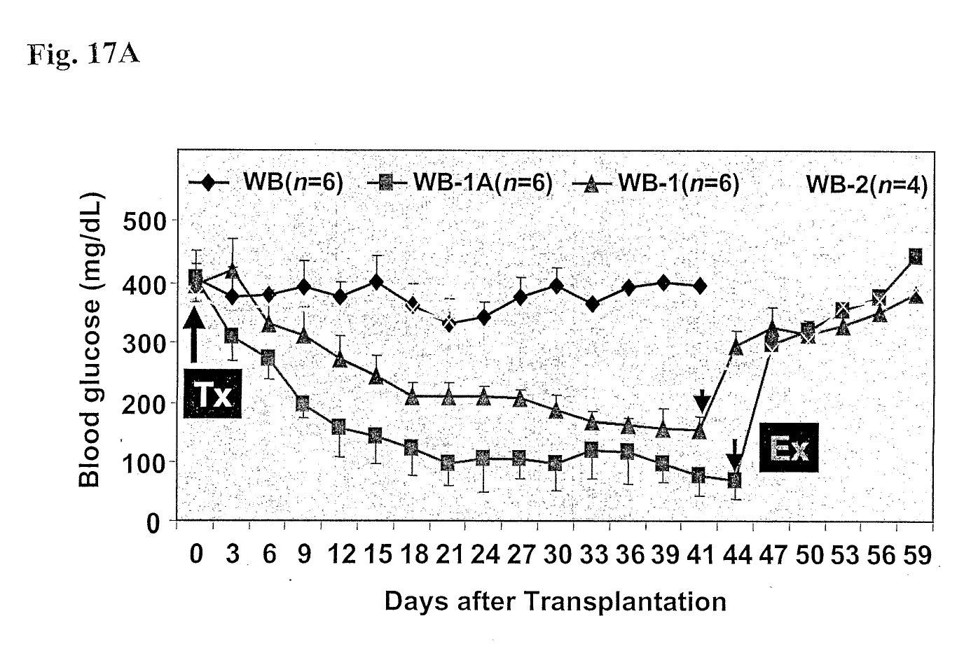

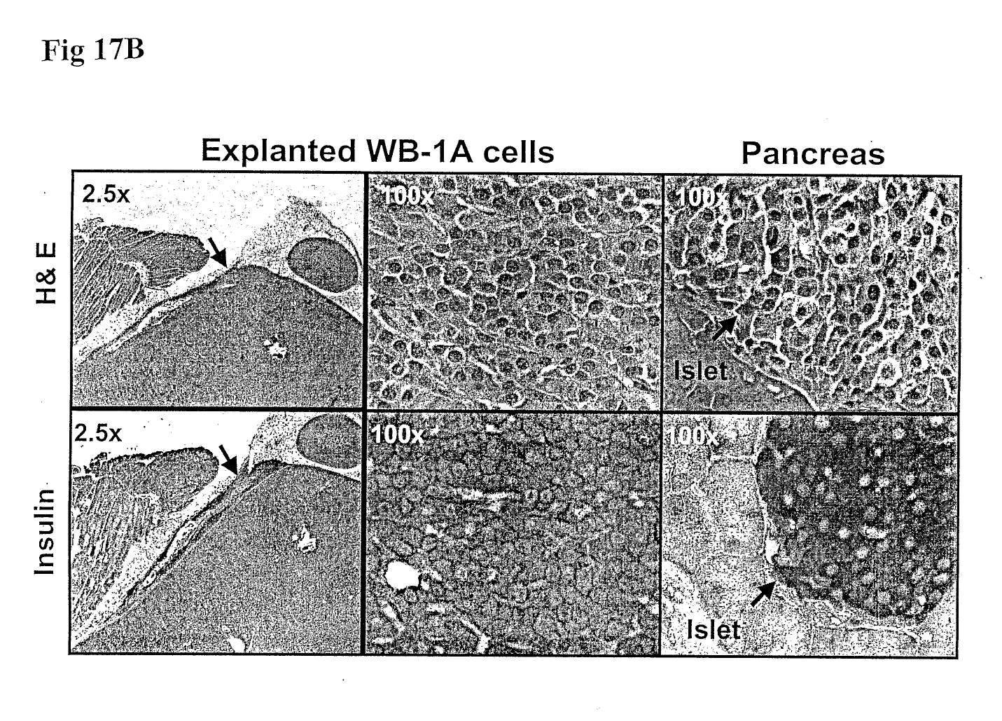

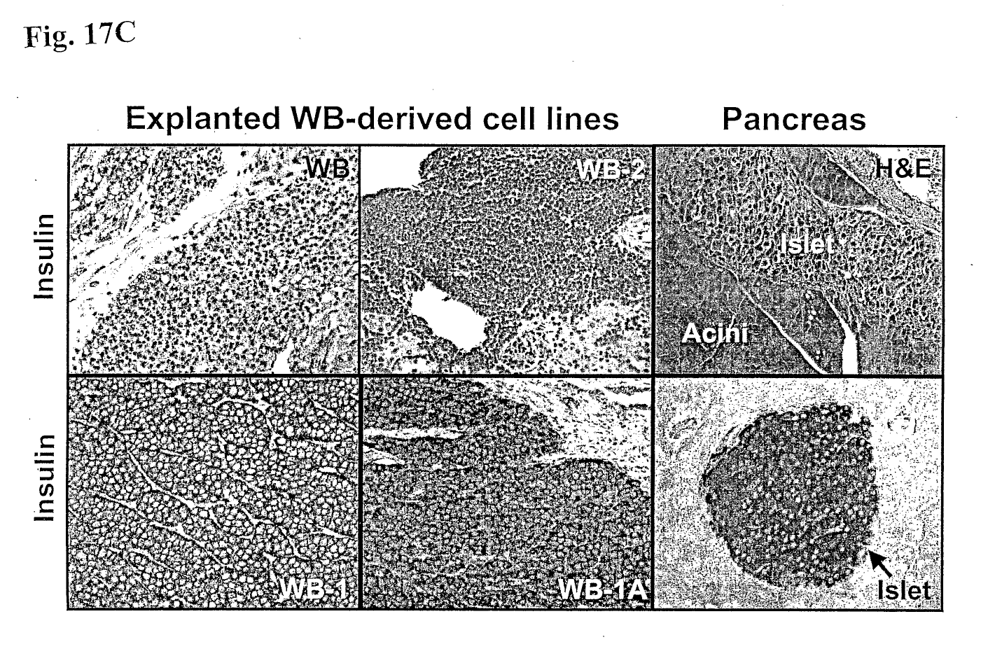

In vivo cell transplantation. The inventors have demonstrated that WB-1 cells can reverse hyperglycemia in diabetic animals, but 2-3 weeks were required for WB-1 cells to undergo in vivo further differentiation and maturation into fully functional β-like cells. To examine the differences in the way that WB-1 and WB-1A cells reverse hyperglycemia, WB-1, WB-1A, WB and WB-2 cells were transplanted into the left renal capsular spaces of Stz-induced diabetic NOD-scid mice. Left nephrectomy was performed around day-40 after transplantation in mice receiving WB-1 and WB-1A cells, and the explanted tissues were analyzed by morphologic and immunologic evaluation. FIG. 17A shows that WB-1 and WB-1A cells are both capable of reducing blood glucose levels in the diabetic mice. However, it took 10 days for the mice transplanted with WB-1A cells to reduce the blood glucose level from ˜400 mg/dL to ˜200 mg/dL, whereas it took around 20 days for the mice transplanted with WB-1 cells to reach the same level. Furthermore, after about 21 days, WB-1A cells completely normalized the blood glucose levels, and maintained the euglycemia up to the time of nephrectomy (square line). The WB-1 cells reduced blood glucose levels to ˜200 mg/dL, but did not normalize blood glucose level (triangle line). Removal of implanted WB-1 and WB-1A cells by left nephrectomy around day-40 post-transplantation induced a persistent rebound hyperglycemia (arrows), confirming that the implanted cells were indeed responsible for the reduction of blood glucose levels. As expected, WB and WB-2 transplanted mice remained hyperglycemia and were sacrificed around day-40 post-transplantation. After two months, WB-1 cells gradually normalized blood glucose level and remained euglycemic (˜100 mg/dL) up to 4 months. To our surprise, the four remaining mice transplanted with WB-1A cells became hypoglycemia (˜40 mg/dL) after two months and died soon thereafter. These results indicate that both WB-1 and WB-LA cells can reverse hyperglycemia with distinct properties of WB-1 cells behaving as β-cell precursors and WB-1A cells as functional mature β-like cells.

Histology and insulin production in explanted cells. To examine morphologic characteristics and insulin production in the explanted cells, the cytological features of WB-1A cells were examined by H&E staining (FIG. 17B, upper) and the amount of insulin by immunostaining (Lower panel). The explanted WB-LA cells exhibited typical neuroendocrine cytology similar to pancreatic islet cells, and contained abundant insulin in more than 95% of the cells with the intensity of insulin staining equivalent to islet β-cells. Additionally, comparison of insulin production between WB-1 and WB-1A cells around day-40 post-transplantation demonstrated that WB-1A cells produced much more insulin (FIG. 17C), which is consistent with their effectiveness in reducing hyperglycemia in diabetic mice. No insulin production was detected in either WB or WB-2 cells (FIG. 17C). These results demonstrate WB-1A cells have the characteristic morphology of mature β-like cells and produce large amounts of insulin, as compared to WB-1 cells at the same time post-transplantation.

Summary: One of the goals of study associated with the invention was to determine whether the transcription factor Pax4, acting alone or in the presence of Pdx1-VP16, could convert rat hepatic stem-like WB cells into functional β-like IPCs in vitro. Activation of Pax4 in WB-1A cells resulted in the activation of multiple downstream late-stage TFs and other genes related to P-cell function and insulin secretion, whereas these genes were silent in the Pdx1-Vp16 expressing pancreatic precursor-like WB-1 cells prior to in vitro or in vivo further differentiation. Dissection of the molecular events occurring in WB-1A cells showed that expression of Pax4 led to the activation of multiple key genes (i.e., Pax-6, MafA, Isl-1, SUR1, Kir6. Z SNAP25, and IAPP), setting the stage for fully differentiated, functional pancreatic β-like cells to emerge. The WB-1A cells produced, processed, and released insulin upon in vitro glucose stimulation, without the need for further in vitro differentiation or in vivo cell transplantation. These cells also exhibited a gene expression profile very similar, if not identical, to that of functional rat INS-1 insulinoma cells. WB-1A cells also produce large amounts of insulin and reversed hyperglycemia in diabetic mice without the typical delay required for in vivo WB-1 cell differentiation. Therefore, coexpression of Pdx1-VP16 and Pax4 in hepatic WB cells can generate functional pancreatic β-like cells.

Little is known about the TF cascade controlling the conversion of hepatic cells into functional β-like IPCs. Introduction of Pdx1-VP16 into hepatic WB cells initiates early-stage P-cell differentiation, resulting in the activation of HNF1, endogenous Pdx1, Ngn3, NeuroD/Beta2, NKx2.2, and NKx6.1 as well as genes related to pancreatic endocrine function (e.g., insulin I & II, glucagon, Glut-2, GK, GLP-R, PC1/3, PC2, HK, and ChromA). However, Pdx1-VP16 alone could not activate such late-stage pancreatic TFs as Pax-4, Pax-6, MafA, and Isl-1, as well as P-cell function-related genes SUR1, Kir6.2, SNAP25, and IAPP in the newly generated WB-1 cells. The aforementioned genes become activated only after further differentiation in vivo when WB-1 cells are transplanted into Stz-induced diabetic NOD-scid mice. The WB-1 cells can also become functional upon long-term in vitro culture in high-glucose medium. The shift of WB-1 cells from glucose-insensitive stage to glucose-responsive phase appears to be triggered by hyperglycemia or high glucose culture condition.

Pax4 is positioned in the upper hierarchy among the silent β-cell transcription factors in WB-1 cells. Its expression in β-cell precursors results in the differentiation and maturation of β-like cells. By inactivating Pax4 in newborn Pax4-deficient mice, the pancreata are almost entirely devoid of mature P-cells, suggesting that Pax4 is crucial for the formation of mature insulin-producing P-cells. However, loss of functional Pax4 does not affect the expression of Ngn3, Islet1, Nkx2.2 or Pax6 in pancreatic precursors. Indeed, as demonstrated herein, forced Pax4 expression in WB-1 cells activated Pax6, Islet1, and MafA, and promoted the cells to undergo further differentiation and maturation. However, overexpression of Pax4 alone in WB-2 cells only activated Ngn3, Isl-1, MafA, and glucagon genes without activating Pax6 and other function-related genes SUR1, Kir6.2, SNAP25, and IAPP, suggesting that Ngn3, Isl-1, MafA, and glucagon genes are direct targets for Pax4 and that the expression of Pax6, SUR1, Kir6.2, SNAP25, and IAPP genes requires participation of other gene in addition to Pax4. It has been reported that Pax4 overexpression in mouse embryonic stem cells reportedly increases IPCs dramatically, as compared to Pdx1-expressing mouse embryonic cells. However, our results indicated that an appropriate background of activated genes related to β-cell development is required for Pax4 to promote β-cell maturation in the context of the liver-to-endocrine pancreas transdifferentiation.

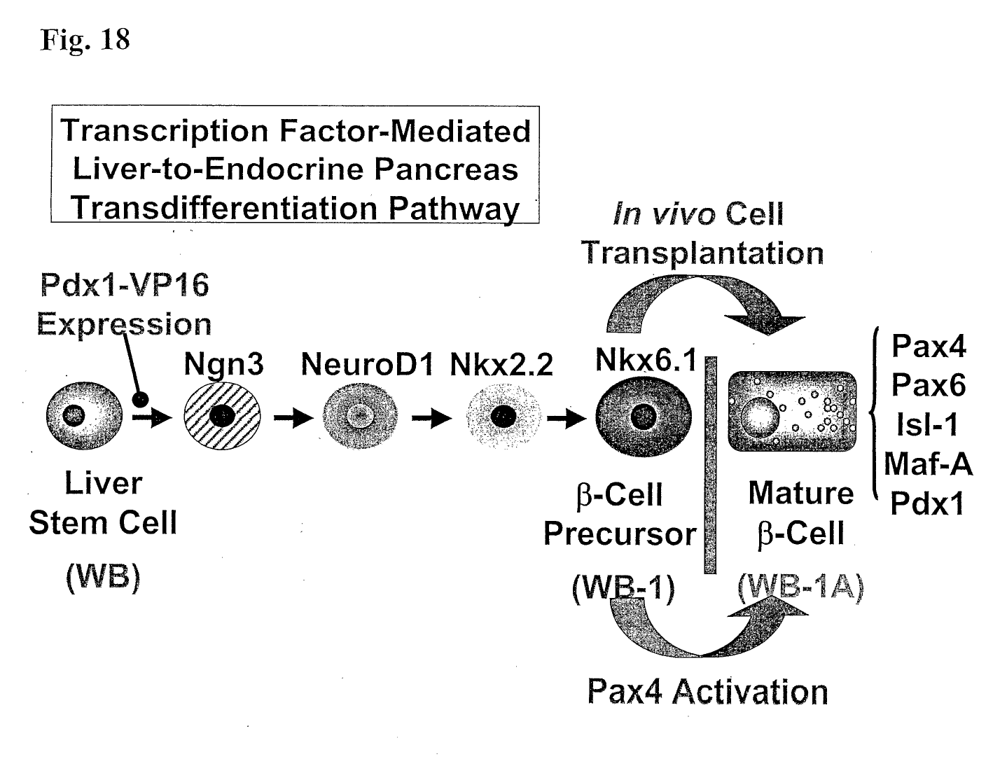

The molecular events for Pdx1-VP16-induced transdifferentiation of hepatic stem-like WB cell into functional insulin-producing β-like cells are summarized schematically in FIG. 18. Expression of Pdx1-VP16 converts the hepatic stem-like WB cells into pancreatic precursor cells characterized by stage-specific TF expression, as illustrated in the figure. However, these precursor cells cannot respond to glucose challenge in vitro without further differentiation. Generation of functional liver-derived pancreatic mature β-like IPCs required: (a) a long-term culture in a high-glucose medium, (b) transplantation of the cells into diabetic mice, or (c) introduction of Pax4 into the Pdx1-VP16-expressing cells. While questions remain concerning the role of Pax4 in the liver-to-endocrine pancreas transdifferentiation, it is clear that Pax4 expression precedes and facilitates Pax6 expression in the Pdx1-VP16-mediated pancreatic transdifferentiation and that constitutive activation of Pax4 significantly down-regulated Nkx2.2 gene expression. The results of previous studies suggest that Nkx6.1 was likely to be an immediate downstream target gene of Pax4 during normal embryogenesis. We find, however, that Nkx6.1 can be activated in the absence of Pax4 expression, suggesting that Nkx6.1 might act upstream of Pax4 in the cascade of TF activation in the liver-to-endocrine pancreas transdifferentiation. Interestingly, a similar gene expression profile (showing Pax4 activation, along with other TFs Pax6, Isl-1, and MafA as well as β-cell function-related genes SUR1, Kir6.2, SNAP25, and IAPP in WB-1 cells) can be reproduced by transplanting WB-1 cells into the diabetic mice. Based on these and other results, it was concluded that expression of both Pdx1-VP16 and Pax4 can convert the hepatic stem-like WB cells into functional liver-derived mature pancreatic β-like IPCs.

The major goal for cell replacement therapy is to generate glucose-regulated insulin-producing cells. Thus, a remaining clinically relevant question is: Is it better to express Pdx1-VP16 alone or together with Pax4 for hepatic cell transdifferentiation for treating type 1 diabetes. Although co-expression of both Pdx1-VP16 and Pax4 can transdifferentiate hepatic WB cells into glucose-responsive cells, our long-term animal studies showed persistent hypoglycemia occurring in WB-LA transplanted mice. Because of Pax4 expression is driven by the strong constitutive EF1α promoter in WB-1A cells, the significant hypoglycemic state in the transplanted mice failed to taper off or to shut down Pax4 expression, even though the immediate upstream Nkx2.2 gene was significantly down-regulated by the persistent expression of Pax4. An alternative approach, therefore, might be to control Pax4 expression with a liver-specific promoter such as transthyretin (TTR) that will shut down the exogenous Pax4 expression upon complete hepatic cell conversion into pancreatic cells or under a glucose-regulatable promoter such as transforming growth factor α. In contrast, Pdx1-VP16 expression reprograms hepatic cells toward the pancreatic endocrine differentiation pathway and generates β-cell precursors. When such cells are transplanted into diabetic animals, euglycemia is maintained until the end of the observation period (4 months). Several important points can be made from the above observations. First, hepatic WB cells should be reprogrammed from a higher position (e.g. Pdx1) in the cascade of β-cell transcription activation, leaving many steps to be regulated by changes in the blood glucose levels. Second, WB-1 cells are in the stage of stem-like β-cell precursors, with some cells remaining at this stage being available to replenish the mature form of β-like cells on an as-needed basis in response to changes of the blood glucose levels. Third, it may be more practical to employ a combination of early and late-stage β-cell TFs to transdifferentiate the liver cells; and the late-stage TF should be controlled by either a glucose-regulatable or a liver-specific promoter.

Overall, the comparative analysis between Pdx1-VP16-expressing WB-1 cells and Pdx1-VP16/Pax4-expressing WB-1A cells suggests that Pax4 expression in WB-1A cells results in a liver-derived functional mature Like phenotype. These cells respond to a high-glucose challenge in vitro or to a hyperglycemia in vivo by releasing insulin. However, they are apparently unable to respond to the lower blood glucose levels in the NOD-scid mice by reducing insulin release due to the constitutive expression of Pax4. Therefore, a more fruitful approach for transdifferentiating liver cells into pancreatic β-like IPCs may be to select suitable gene(s) from a higher position in the cascade of transcriptional control of P-cell development and/or suitable glucose regulatable or liver-specific promoters. This selection might fulfill the requirement for selective hepatic cell transdifferentiation into pancreatic β-like insulin-producing cells, while, at the same time, allowing for the genetically engineered β-like cells to respond effectively to the changes in blood glucose levels.

TABLE 1
List of primer information for RT-PCR

| | | | Size of | | | |
|---|---|---|---|---|---|---|
| | | | PCR | | | |
| | | | Product | GenBank | Tm | Cycles |
| Genes | Forward primer | Reverse primer | (bp) | Acc. No. | (° C.) | (#) |
| Actin | cgt aaa gac ctc tat gcc aa | agc cat gcc aaa tgt ctc at | 351 | V01217 | 56 | 35 |
| HNF-1 | ttc taa gct gag cca gct gca | gct gag gtt ctc cgg ctc ttt | 275 | X54423 | 56 | 35 |
| | gac g | cag a | | | | |
| MPdx1 | tac aag ctc gct ggg atc act | gca gta cgg gtc ctc ttg tt | 309 | X_74342 | 56 | 35 |
| rPdx1 | cgg cca cac agc tct aca agg | gag gtt acg gca caa tcc tgc | 667 | NM_022852 | 56 | 35 |
| Ngn3 | ctg cgc ata gcg gac cac agc | ctt cac aag aag tct gag aac | 324 | NM_021700 | 58 | 35 |
| | ttc | acc ag | | | | |
| NeuroD | ctt ggc caa gaa cta cat ctg | gga gta ggg atg cac cgg gaa | 225 | NM_019218 | 57 | 35 |
| | g | | | | | |
| Nkx2.2 | Gtacacgcgctggctggccag | gtacacgcgctggctggccag | 304 | NM_010919 | 56 | 35 |
| Pax4 | cag cag cat gga cca gct tgg | ctc ctg taa tgc ccg cag gac | 214 | XM_133023 | 55 | 35 |
| Nkx6.1 | atg gga aga gaa aac aca cca | taa tcg tcg tcg tcc tcc tcg | 280 | AF357883 | 58 | 35 |
| | gac | ttc | | | | |
| Pax6 | gag aca gat tac tct ccg agg | acc aca cct gta tcc ttg ctt | 465 | NM_013001 | 55 | 35 |
| | | ag g | | | | |
| Isl1 | cgg gag gat ggg ctt ttc tg | agc tgc ttt tgg ttg agc aca | 191 | NM_017339 | 56 | 35 |
| | | g | | | | |
| MafA | gac atc tcc cca tac gaa gtg | ccg cta cta cgt ttc tta tct | 462 | NM_008814 | 55 | 35 |
| Glut-2 | tcc agt aca ttg cgg act tcc | ggt gta gtc cta cac tca tg | 304 | J03145 | 58 | 35 |
| GK | aag gga aca aca tcg tag ga | cta tgg cgg tct tca tag ta | 126 | X53569 | 56 | 35 |
| Insulin I | tac aat cat aga cca tca gca | cag ttg gta gag gga gca gat | 355 | Gi:204956 | 56 | 35 |
| Insulin II | agc cct aag tga cca gct aca | tgc caa ggt ctg aag gtc ac | 343 | V01243 | 56 | 35 |
| PP | gtc gca tac tac tgc ctc tcc | aga cag aag gga ggc tac aaa | 336 | NM_012626 | 57 | 35 |
| | | tcc | | | | |
| Glucagon | gac cgt tta cat cgt ggc gg | cgg ttc ctc ttg gtg ttc atc | 249 | NM_012707 | 58 | 35 |
| | | aac | | | | |
| Somatostatin | atg ctg tcc tgc cgt ctc c | tcg agt tgg cag acc tct g | 277 | NM_012659 | 56 | 35 |
| GLP-1R | tct ctt ctg caa ccg aac ct | ctg gtg cag tgc aag tgt ct | 351 | S75952 | 58 | 35 |
| SUR-1 | Aag atc atg cac ttg tct act | aga cag cag gaa cag cgg tgt | 593 | AF039595 | 55 | 35 |
| SNAP-25 | Agt agt ggc cag cca gcc tg | atc tgg cga ttc tgg gtg tca | 200 | NM_030991 | 57 | 35 |
| Hexokinase | Tga acc acg aga aga acc aga | Aca atg tta gca tca tag tcc | 322 | NM_012734 | 58 | 35 |
| PC-1 | Ttt gtc agt atg cgt gct aac | Ctg tga cga tgc tgt aat gat | 554 | AB071596 | 58 | 35 |
| PC-2 | Agg tgg tga ggg att acc aa | Aga act gtg gac caa gga ga | 177 | NM_012746 | 58 | 35 |
| Kir6.2 | Acc acg ctg gtg gac ctc aag | Gca cca cct gca tat gaa tgg | 481 | RNU44897 | 60 | 35 |
| IAPP | Ggc tgt agt tcc tga agc tt | aag gtt gtt gct gga gcg aa | 260 | NM_012586 | 56 | 35 |
| chromogranin A | Act aag gtg atg aag tgt gt | tct cta cag tgt cct tgg ag | 353 | NM_021655 | 56 | 35 |

TABLE 2
Insulin content and insulin release during 2 hours after glucose stimulation.

| | Insulin content | Insulin release (ng/mg protein/2 h) | | Fold of insulin release |
|---|---|---|---|---|
| Cell | (ng/mg protein) | 20 mM Glucose (−) | 20 mM Glucose (+) | 20 mM Glucose (+)/(−) |
| WB | 0.26 ± 0.01* | 0.40 ± 0.01* | 0.39 ± 0.01* | 0.98 |
| WB-1 | 2.73 ± 0.01 | 0.79 ± 0.02 | 0.84 ± 0.02 | 1.06 |
| WB-2 | 0.50 ± 0.02* | 0.47 ± 0.01* | 0.47 ± 0.02* | 1.00 |
| WB-1A | 22.0 ± 0.13 | 1.66 ± 0.01 | 3.38 ± 0.05*** | 2.04 |

*No statistical significance from blank or cell culture medium readings
***P < 0.001.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for making an insulin-producing cell comprising the steps of:
providing a liver cell, and increasing a level of at least two transcription factors in the liver cell under conditions that promote the selective differentiation of the liver cell into an insulin-producing cell.

2. The method of claim 1, wherein said two transcription factors comprise Pdx-1 and Pax4.

3. The method of claim 2, wherein the liver cell is contacted with a first vector comprising a nucleic acid encoding said Pdx-1-VP16 and a second vector comprising a nucleic acid encoding said Pax4.

4. The method of claim 3, wherein the first vector and the second vector comprise nucleic acid sequences derived from a lentivirus.

5. An insulin-producing cell made according to the method of claim 1.

6. A method of reducing insulin insufficiency in a subject, comprising the step of introducing into the subject a composition selected from the group consisting of the cell of claim 5 and an agent that causes an increase in the level of at least two transcription factors in one or more target (e.g., liver) cells in the subject.

7. The method of claim 6, wherein said two transcription factors comprise Pdx-1 and Pax4.

8. The method of claim 7, wherein the liver cell is contacted with a first vector comprising a nucleic acid encoding said Pdx-1-VP16 and a second vector comprising a nucleic acid encoding said Pax4.

9. The method of claim 8, wherein the first vector and the second vector comprise nucleic acid sequences derived from a lentivirus.

10. The method of claim 6, wherein the cell of claim 5 is not derived from the subject.

11. The method of claim 6, wherein the cell of claim 5 is derived from the subject.

12. The method of claim 6, wherein the cell of claim 5 is introduced into the subject by implantation into a target tissue or organ.

13. The method of claim 12, wherein the target tissue or organ is a liver.

14. An insulin producing cell comprising an expression vector encoding Pdx1-VP16 and Pax4.

15. The insulin producing cell of claim 14, wherein Pdx1 and Pax4 are expressed in the cell.

16. The insulin producing cell of claim 15, wherein co-expression of Pdx1 and Pax4 by said cell differentiates into a functional pancreatic β-like cell.

17. The insulin-producing cell of claim 15, wherein said cell is a hepatic stem cell.
